SEQUENCE LISTING (73 sequences)

SEQ ID NO: 1 - protein, 903 aa, Homo sapiens
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSENGVSLCLPRLECNSAISAHCNLCLPGLSDSPISASRVAGFTGASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 2 - protein, 66 aa, Artificial Sequence
  Synthetic
APLPPGWEQRVDQHGRVYYVDHVEKRTTWDRPEPLPPGWERRVDNMGRIYYVDHFTRTTTWQRPTL

SEQ ID NO: 3 - protein, 46 aa, Artificial Sequence
  Synthetic
ESVRNYEQWQLQRSQLQGAMQQFNQRFIYGNQDLFATSQSKEFDPL

SEQ ID NO: 4 - protein, 34 aa, Artificial Sequence
  Synthetic
GPLPPGWEKRTDSNGRVYFVNHNTRITQWEDPRS

SEQ ID NO: 5 - protein, 6 aa, Artificial Sequence
  Synthetic
QGQLNE

SEQ ID NO: 6 - protein, 34 aa, Artificial Sequence
  Synthetic
KPLPEGWEMRFTVDGIPYFVDHNRRTTTYIDPRT

SEQ ID NO: 7 - protein, 222 aa, Artificial Sequence
  Synthetic
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 8 - protein, 104 aa, Artificial Sequence
  Synthetic
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFA

SEQ ID NO: 9 - protein, 75 aa, Artificial Sequence
  Synthetic
MYSFVSEETGTLIVNSVLLFLAFVVFLLVTLAILTALRLCAYCCNIVNVSLVKPSFYVYSRVKNLNSSRVPDLLV

SEQ ID NO: 10 - protein, 1273 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11-12 - skipped (2 entries)

SEQ ID NO: 13 - protein, 34 aa, Artificial Sequence
  Synthetic
APLPPGWEQRVDQHGRVYYVDHVEKRTTWDRPEP

SEQ ID NO: 14 - protein, 34 aa, Artificial Sequence
  Synthetic
EPLPPGWERRVDNMGRIYYVDHFTRTTTWQRPTL

SEQ ID NO: 15 - protein, 347 aa, Homo sapiens
MAQSRDGGNPFAEPSELDNPFQDPAVIQHRPSRQYATLDVYNPFETREPPPAYEPPAPAPLPPPSAPSLQPSRKLSPTEPKNYGSYSTQASAAAATAELLKKQEELNRKAEELDRRERELQHAALGGTATRQNNWPPLPSFCPVQPCFFQDISMEIPQEFQKTVSTMYYLWMCSTLALLLNFLACLASFCVETNNGAGFGLSILWVLLFTPCSFVCWYRPMYKAFRSDSSFNFFVFFFIFFVQDVLFVLQAIGIPGWGFSGWISALVVPKGNTAVSVLMLLVALLFTGIAVLGIVMLKRIHSLYRRTGASFQKAQQEFAAGVFSNPAVRTAAANAAAGAAENAFRAP

SEQ ID NO: 16 - protein, 4 aa, Artificial Sequence
  Synthetic
PPAY

SEQ ID NO: 17 - protein, 4 aa, Artificial Sequence
  Synthetic
PSAP

SEQ ID NO: 18 - protein, 21 aa, Artificial Sequence
  Synthetic
WMCSTLALLLNFLACLASFCV

SEQ ID NO: 19 - protein, 21 aa, Artificial Sequence
  Synthetic
AGFGLSILWVLLFTPCSFVCW

SEQ ID NO: 20 - protein, 21 aa, Artificial Sequence
  Synthetic
FVLQAIGIPGWGFSGWISALV

SEQ ID NO: 21 - protein, 21 aa, Artificial Sequence
  Synthetic
VLMLLVALLFTGIAVLGIVML

SEQ ID NO: 22 - protein, 4 aa, Artificial Sequence
  Synthetic
  misc_feature (3)..(3): Xaa can be any naturally occurring amino acid
PPXY

SEQ ID NO: 23-24 - skipped (2 entries)

SEQ ID NO: 25 - protein, 922 aa, Homo sapiens
MATASPRSDTSNNHSGRLQLQVTVSSAKLKRKKNWFGTAIYTEVVVDGEITKTAKSSSSSNPKWDEQLTVNVTPQTTLEFQVWSHRTLKADALLGKATIDLKQALLIHNRKLERVKEQLKLSLENKNGIAQTGELTVVLDGLVIEQENITNCSSSPTIEIQENGDALHENGEPSARTTARLAVEGTNGIDNHVPTSTLVQNSCCSYVVNGDNTPSSPSQVAARPKNTPAPKPLASEPADDTVNGESSSFAPTDNASVTGTPVVSEENALSPNCTSTTVEDPPVQEILTSSENNECIPSTSAELESEARSILEPDTSNSRSSSAFEAAKSRQPDGCMDPVRQQSGNANTETLPSGWEQRKDPHGRTYYVDHNTRTTTWERPQPLPPGWERRVDDRRRVYYVDHNTRTTTWQRPTMESVRNFEQWQSQRNQLQGAMQQFNQRYLYSASMLAAENDPYGPLPPGWEKRVDSTDRVYFVNHNTKTTQWEDPRTQGLQNEEPLPEGWEIRYTREGVRYFVDHNTRTTTFKDPRNGKSSVTKGGPQIAYERGFRWKLAHFRYLCQSNALPSHVKINVSRQTLFEDSFQQIMALKPYDLRRRLYVIFRGEEGLDYGGLAREWFFLLSHEVLNPMYCLFEYAGKNNYCLQINPASTINPDHLSYFCFIGRFIAMALFHGKFIDTGFSLPFYKRMLSKKLTIKDLESIDTEFYNSLIWIRDNNIEECGLEMYFSVDMEILGKVTSHDLKLGGSNILVTEENKDEYIGLMTEWRFSRGVQEQTKAFLDGFNEVVPLQWLQYFDEKELEVMLCGMQEVDLADWQRNTVYRHYTRNSKQIIWFWQFVKETDNEVRMRLLQFVTGTCRLPLGGFAELMGSNGPQKFCIEKVGKDTWLPRSHTCFNRLDLPPYKSYEQLKEKLLFAIEETEGFGQE

SEQ ID NO: 26 - protein, 34 aa, Artificial Sequence
  Synthetic
ETLPSGWEQRKDPHGRTYYVDHNTRTTTWERPQP

SEQ ID NO: 27 - protein, 34 aa, Artificial Sequence
  Synthetic
QPLPPGWERRVDDRRRVYYVDHNTRTTTWQRPTM

SEQ ID NO: 28 - protein, 39 aa, Artificial Sequence
  Synthetic
ENDPYGPLPPGWEKRVDSTDRVYFVNHNTKTTQWEDPRT

SEQ ID NO: 29 - protein, 34 aa, Artificial Sequence
  Synthetic
EPLPEGWEIRYTREGVRYFVDHNTRTTTFKDPRN

SEQ ID NO: 30 - protein, 870 aa, Homo sapiens
MASASSSRAGVALPFEKSQLTLKVVSAKPKVHNRQPRINSYVEVAVDGLPSETKKTGKRIGSSELLWNEIIILNVTAQSHLDLKVWSCHTLRNELLGTASVNLSNVLKNNGGKMENMQLTLNLQTENKGSVVSGGELTIFLDGPTVDLGNVPNGSALTDGSQLPSRDSSGTAVAPENRHQPPSTNCFGGRSRTHRHSGASARTTPATGEQSPGARSRHRQPVKNSGHSGLANGTVNDEPTTATDPEEPSVVGVTSPPAAPLSVTPNPNTTSLPAPATPAEGEEPSTSGTQQLPAAAQAPDALPAGWEQRELPNGRVYYVDHNTKTTTWERPLPPGWEKRTDPRGRFYYVDHNTRTTTWQRPTAEYVRNYEQWQSQRNQLQGAMQHFSQRFLYQSSSASTDHDPLGPLPPGWEKRQDNGRVYYVNHNTRTTQWEDPRTQGMIQEPALPPGWEMKYTSEGVRYFVDHNTRTTTFKDPRPGFESGTKQGSPGAYDRSFRWKYHQFRFLCHSNALPSHVKISVSRQTLFEDSFQQIMNMKPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELYFIQDMEILGKVTTHELKEGGESIRVTEENKEEYIMLLTDWRFTRGVEEQTKAFLDGFNEVAPLEWLRYFDEKELELMLCGMQEIDMSDWQKSTIYRHYTKNSKQIQWFWQVVKEMDNEKRIRLLQFVTGTCRLPVGGFAELIGSNGPQKFCIDKVGKETWLPRSHTCFNRLDLPPYKSYEQLREKLLYAIEETEGFGQE

SEQ ID NO: 31 - protein, 34 aa, Artificial Sequence
  Synthetic
DALPAGWEQRELPNGRVYYVDHNTKTTTWERPLP

SEQ ID NO: 32 - protein, 33 aa, Artificial Sequence
  Synthetic
PLPPGWEKRTDPRGRFYYVDHNTRTTTWQRPTA

SEQ ID NO: 33 - protein, 37 aa, Artificial Sequence
  Synthetic
HDPLGPLPPGWEKRQDNGRVYYVNHNTRTTQWEDPRT

SEQ ID NO: 34 - protein, 34 aa, Artificial Sequence
  Synthetic
PALPPGWEMKYTSEGVRYFVDHNTRTTTFKDPRP

SEQ ID NO: 35 - protein, 1319 aa, Homo sapiens
MAQSLRLHFAARRSNTYPLSETSGDDLDSHVHMCFKRPTRISTSNVVQMKLTPRQTALAPLIKENVQSQERSSVPSSENVNKKSSCLQISLQPTRYSGYLQSSNVLADSDDASFTCILKDGIYSSAVVDNELNAVNDGHLVSSPAICSGSLSNFSTSDNGSYSSNGSDFGSCASITSGGSYTNSVISDSSSYTFPPSDDTFLGGNLPSDSTSNRSVPNRNTTPCEIFSRSTSTDPFVQDDLEHGLEIMKLPVSRNTKIPLKRYSSLVIFPRSPSTTRPTSPTSLCTLLSKGSYQTSHQFIISPSEIAHNEDGTSAKGFLSTAVNGLRLSKTICTPGEVRDIRPLHRKGSLQKKIVLSNNTPRQTVCEKSSEGYSCVSVHFTQRKAATLDCETTNGDCKPEMSEIKLNSDSEYIKLMHRTSACLPSSQNVDCQININGELERPHSQMNKNHGILRRSISLGGAYPNISCLSSLKHNCSKGGPSQLLIKFASGNEGKVDNLSRDSNRDCTNELSNSCKTRDDFLGQVDVPLYPLPTENPRLERPYTFKDFVLHPRSHKSRVKGYLRLKMTYLPKTSGSEDDNAEQAEELEPGWVVLDQPDAACHLQQQQEPSPLPPGWEERQDILGRTYYVNHESRRTQWKRPTPQDNLTDAENGNIQLQAQRAFTTRRQISEETESVDNRESSENWEIIREDEATMYSNQAFPSPPPSSNLDVPTHLAEELNARLTIFGNSAVSQPASSSNHSSRRGSLQAYTFEEQPTLPVLLPTSSGLPPGWEEKQDERGRSYYVDHNSRTTTWTKPTVQATVETSQLTSSQSSAGPQSQASTSDSGQQVTQPSEIEQGFLPKGWEVRHAPNGRPFFIDHNTKTTTWEDPRLKIPAHLRGKTSLDTSNDLGPLPPGWEERTHTDGRIFYINHNIKRTQWEDPRLENVAITGPAVPYSRDYKRKYEFFRRKLKKQNDIPNKFEMKLRRATVLEDSYRRIMGVKRADFLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLHKPITLHDMESVDSEYYNSLRWILENDPTELDLRFIIDEELFGQTHQHELKNGGSEIVVTNKNKKEYIYLVIQWRFVNRIQKQMAAFKEGFFELIPQDLIKIFDENELELLMCGLGDVDVNDWREHTKYKNGYSANHQVIQWFWKAVLMMDSEKRIRLLQFVTGTSRVPMNGFAELYGSNGPQSFTVEQWGTPEKLPRAHTCFNRLDLPPYESFEELWDKLQMAIENTQGFDGVD

SEQ ID NO: 36 - protein, 34 aa, Artificial Sequence
  Synthetic
SPLPPGWEERQDILGRTYYVNHESRRTQWKRPTP

SEQ ID NO: 37 - protein, 34 aa, Artificial Sequence
  Synthetic
SGLPPGWEEKQDERGRSYYVDHNSRTTTWTKPTV

SEQ ID NO: 38 - protein, 34 aa, Artificial Sequence
  Synthetic
GFLPKGWEVRHAPNGRPFFIDHNTKTTTWEDPRL

SEQ ID NO: 39 - protein, 34 aa, Artificial Sequence
  Synthetic
GPLPPGWEERTHTDGRIFYINHNIKRTQWEDPRL

SEQ ID NO: 40 - protein, 955 aa, Homo sapiens
MATGLGEPVYGLSEDEGESRILRVKVVSGIDLAKKDIFGASDPYVKLSLYVADENRELALVQTKTIKKTLNPKWNEEFYFRVNPSNHRLLFEVFDENRLTRDDFLGQVDVPLSHLPTEDPTMERPYTFKDFLLRPRSHKSRVKGFLRLKMAYMPKNGGQDEENSDQRDDMEHGWEVVDSNDSASQHQEELPPPPLPPGWEEKVDNLGRTYYVNHNNRTTQWHRPSLMDVSSESDNNIRQINQEAAHRRFRSRRHISEDLEPEPSEGGDVPEPWETISEEVNIAGDSLGLALPPPPASPGSRTSPQELSEELSRRLQITPDSNGEQFSSLIQREPSSRLRSCSVTDAVAEQGHLPPPSVAYVHTTPGLPSGWEERKDAKGRTYYVNHNNRTTTWTRPIMQLAEDGASGSATNSNNHLIEPQIRRPRSLSSPTVTLSAPLEGAKDSPVRRAVKDTLSNPQSPQPSPYNSPKPQHKVTQSFLPPGWEMRIAPNGRPFFIDHNTKTTTWEDPRLKFPVHMRSKTSLNPNDLGPLPPGWEERIHLDGRTFYIDHNSKITQWEDPRLQNPAITGPAVPYSREFKQKYDYFRKKLKKPADIPNRFEMKLHRNNIFEESYRRIMSVKRPDVLKARLWIEFESEKGLDYGGVAREWFFLLSKEMFNPYYGLFEYSATDNYTLQINPNSGLCNEDHLSYFTFIGRVAGLAVFHGKLLDGFFIRPFYKMMLGKQITLNDMESVDSEYYNSLKWILENDPTELDLMFCIDEENFGQTYQVDLKPNGSEIMVTNENKREYIDLVIQWRFVNRVQKQMNAFLEGFTELLPIDLIKIFDENELELLMCGLGDVDVNDWRQHSIYKNGYCPNHPVIQWFWKAVLLMDAEKRIRLLQFVTGTSRVPMNGFAELYGSNGPQLFTIEQWGSPEKLPRAHTCFNRLDLPPYETFEDLREKLLMAVENAQGFEGVD

SEQ ID NO: 41 - protein, 27 aa, Artificial Sequence
  Synthetic
GWEEKVDNLGRTYYVNHNNRTTQWHRP

SEQ ID NO: 42 - protein, 29 aa, Artificial Sequence
  Synthetic
PSGWEERKDAKGRTYYVNHNNRTTTWTRP

SEQ ID NO: 43 - protein, 31 aa, Artificial Sequence
  Synthetic
PPGWEMRIAPNGRPFFIDHNTKTTTWEDPRL

SEQ ID NO: 44 - protein, 31 aa, Artificial Sequence
  Synthetic
PPGWEERIHLDGRTFYIDHNSKITQWEDPRL

SEQ ID NO: 45 - protein, 757 aa, Homo sapiens
MSNPGTRRNGSSIKIRLTVLCAKNLAKKDFFRLPDPFAKIVVDGSGQCHSTDTVKNTLDPKWNQHYDLYVGKTDSITISVWNHKKIHKKQGAGFLGCVRLLSNAISRLKDTGYQRLDLCKLNPSDTDAVRGQIVVSLQTRDRIGTGGSVVDCRGLLENEGTVYEDSGPGRPLSCFMEEPAPYTDSTGAAAGGGNCRFVESPSQDQRLQAQRLRNPDVRGSLQTPQNRPHGHQSPELPEGYEQRTTVQGQVYFLHTQTGVSTWHDPRIPSPSGTIPGGDAAFLYEFLLQGHTSEPRDLNSVNCDELGPLPPGWEVRSTVSGRIYFVDHNNRTTQFTDPRLHHIMNHQCQLKEPSQPLPLPSEGSLEDEELPAQRYERDLVQKLKVLRHELSLQQPQAGHCRIEVSREEIFEESYRQIMKMRPKDLKKRLMVKFRGEEGLDYGGVAREWLYLLCHEMLNPYYGLFQYSTDNIYMLQINPDSSINPDHLSYFHFVGRIMGLAVFHGHYINGGFTVPFYKQLLGKPIQLSDLESVDPELHKSLVWILENDITPVLDHTFCVEHNAFGRILQHELKPNGRNVPVTEENKKEYVRLYVNWRFMRGIEAQFLALQKGFNELIPQHLLKPFDQKELELIIGGLDKIDLNDWKSNTRLKHCVADSNIVRWFWQAVETFDEERRARLLQFVTGSTRVPLQGFKALQGSTGAAGPRLFTIHLIDANTDNLPKAHTCFNRIDIPPYESYEKLYEKLLTAVEETCGFAVE

SEQ ID NO: 46 - protein, 34 aa, Artificial Sequence
  Synthetic
PELPEGYEQRTTVQGQVYFLHTQTGVSTWHDPRI

SEQ ID NO: 47 - protein, 34 aa, Artificial Sequence
  Synthetic
GPLPPGWEVRSTVSGRIYFVDHNNRTTQFTDPRL

SEQ ID NO: 48 - protein, 748 aa, Homo sapiens
MSNPGGRRNGPVKLRLTVLCAKNLVKKDFFRLPDPFAKVVVDGSGQCHSTDTVKNTLDPKWNQHYDLYIGKSDSVTISVWNHKKIHKKQGAGFLGCVRLLSNAINRLKDTGYQRLDLCKLGPNDNDTVRGQIVVSLQSRDRIGTGGQVVDCSRLFDNDLPDGWEERRTASGRIQYLNHITRTTQWERPTRPASEYSSPGRPLSCFVDENTPISGTNGATCGQSSDPRLAERRVRSQRHRNYMSRTHLHTPPDLPEGYEQRTTQQGQVYFLHTQTGVSTWHDPRVPRDLSNINCEELGPLPPGWEIRNTATGRVYFVDHNNRTTQFTDPRLSANLHLVLNRQNQLKDQQQQQVVSLCPDDTECLTVPRYKRDLVQKLKILRQELSQQQPQAGHCRIEVSREEIFEESYRQVMKMRPKDLWKRLMIKFRGEEGLDYGGVAREWLYLLSHEMLNPYYGLFQYSRDDIYTLQINPDSAVNPEHLSYFHFVGRIMGMAVFHGHYIDGGFTLPFYKQLLGKSITLDDMELVDPDLHNSLVWILENDITGVLDHTFCVEHNAYGEIIQHELKPNGKSIPVNEENKKEYVRLYVNWRFLRGIEAQFLALQKGFNEVIPQHLLKTFDEKELELIICGLGKIDVNDWKVNTRLKHCTPDSNIVKWFWKAVEFFDEERRARLLQFVTGSSRVPLQGFKALQGAAGPRLFTIHQIDACTNNLPKAHTCFNRIDIPPYESYEKLYEKLLTAIEETCGFAVE

SEQ ID NO: 49 - protein, 34 aa, Artificial Sequence
  Synthetic
NDLPDGWEERRTASGRIQYLNHITRTTQWERPTR

SEQ ID NO: 50 - protein, 34 aa, Artificial Sequence
  Synthetic
PDLPEGYEQRTTQQGQVYFLHTQTGVSTWHDPRV

SEQ ID NO: 51 - protein, 34 aa, Artificial Sequence
  Synthetic
GPLPPGWEIRNTATGRVYFVDHNNRTTQFTDPRL

SEQ ID NO: 52 - protein, 1606 aa, Homo sapiens
MLLHLCSVKNLYQNRFLGLAAMASPSRNSQSRRRCKEPLRYSYNPDQFHNMDLRGGPHDGVTIPRSTSDTDLVTSDSRSTLMVSSSYYSIGHSQDLVIHWDIKEEVDAGDWIGMYLIDEVLSENFLDYKNRGVNGSHRGQIIWKIDASSYFVEPETKICFKYYHGVSGALRATTPSVTVKNSAAPIFKSIGADETVQGQGSRRLISFSLSDFQAMGLKKGMFFNPDPYLKISIQPGKHSIFPALPHHGQERRSKIIGNTVNPIWQAEQFSFVSLPTDVLEIEVKDKFAKSRPIIKRFLGKLSMPVQRLLERHAIGDRVVSYTLGRRLPTDHVSGQLQFRFEITSSIHPDDEEISLSTEPESAQIQDSPMNNLMESGSGEPRSEAPESSESWKPEQLGEGSVPDGPGNQSIELSRPAEEAAVITEAGDQGMVSVGPEGAGELLAQVQKDIQPAPSAEELAEQLDLGEEASALLLEDGEAPASTKEEPLEEEATTQSRAGREEEEKEQEEEGDVSTLEQGEGRLQLRASVKRKSRPCSLPVSELETVIASACGDPETPRTHYIRIHTLLHSMPSAQGGSAAEEEDGAEEESTLKDSSEKDGLSEVDTVAADPSALEEDREEPEGATPGTAHPGHSGGHFPSLANGAAQDGDTHPSTGSESDSSPRQGGDHSCEGCDASCCSPSCYSSSCYSTSCYSSSCYSASCYSPSCYNGNRFASHTRFSSVDSAKISESTVFSSQDDEEEENSAFESVPDSMQSPELDPESTNGAGPWQDELAAPSGHVERSPEGLESPVAGPSNRREGECPILHNSQPVSQLPSLRPEHHHYPTIDEPLPPNWEARIDSHGRVFYVDHVNRTTTWQRPTAAATPDGMRRSGSIQQMEQLNRRYQNIQRTIATERSEEDSGSQSCEQAPAGGGGGGGSDSEAESSQSSLDLRREGSLSPVNSQKITLLLQSPAVKFITNPEFFTVLHANYSAYRVFTSSTCLKHMILKVRRDARNFERYQHNRDLVNFINMFADTRLELPRGWEIKTDQQGKSFFVDHNSRATTFIDPRIPLQNGRLPNHLTHRQHLQRLRSYSAGEASEVSRNRGASLLARPGHSLVAAIRSQHQHESLPLAYNDKIVAFLRQPNIFEMLQERQPSLARNHTLREKIHYIRTEGNHGLEKLSCDADLVILLSLFEEEIMSYVPLQAAFHPGYSFSPRCSPCSSPQNSPGLQRASARAPSPYRRDFEAKLRNFYRKLEAKGFGQGPGKIKLIIRRDHLLEGTFNQVMAYSRKELQRNKLYVTFVGEEGLDYSGPSREFFFLLSQELFNPYYGLFEYSANDTYTVQISPMSAFVENHLEWFRFSGRILGLALIHQYLLDAFFTRPFYKALLRLPCDLSDLEYLDEEFHQSLQWMKDNNITDILDLTFTVNEEVFGQVTERELKSGGANTQVTEKNKKEYIERMVKWRVERGVVQQTEALVRGFYEVVDSRLVSVFDARELELVIAGTAEIDLNDWRNNTEYRGGYHDGHLVIRWFWAAVERFNNEQRLRLLQFVTGTSSVPYEGFAALRGSNGLRRFCIEKWGKITSLPRAHTCFNRLDLPPYPSYSMLYEKLLTAVEETSTFGLE

SEQ ID NO: 53 - protein, 33 aa, Artificial Sequence
  Synthetic
PLPPNWEARIDSHGRVFYVDHVNRTTTWQRPTA

SEQ ID NO: 54 - protein, 34 aa, Artificial Sequence
  Synthetic
LELPRGWEIKTDQQGKSFFVDHNSRATTFIDPRI

SEQ ID NO: 55 - protein, 1572 aa, Homo sapiens
MASSAREHLLFVRRRNPQMRYTLSPENLQSLAAQSSMPENMTLQRANSDTDLVTSESRSSLTASMYEYTLGQAQNLIIFWDIKEEVDPSDWIGLYHIDENSPANFWDSKNRGVTGTQKGQIVWRIEPGPYFMEPEIKICFKYYHGISGALRATTPCITVKNPAVMMGAEGMEGGASGNLHSRKLVSFTLSDLRAVGLKKGMFFNPDPYLKMSIQPGKKSSFPTCAHHGQERRSTIISNTTNPIWHREKYSFFALLTDVLEIEIKDKFAKSRPIIKRFLGKLTIPVQRLLERQAIGDQMLSYNLGRRLPADHVSGYLQFKVEVTSSVHEDASPEAVGTILGVNSVNGDLGSPSDDEDMPGSHHDSQVCSNGPVSEDSAADGTPKHSFRTSSTLEIDTEELTSTSSRTSPPRGRQDSLNDYLDAIEHNGHSRPGTATCSERSMGASPKLRSSFPTDTRLNAMLHIDSDEEDHEFQQDLGYPSSLEEEGGLIMFSRASRADDGSLTSQTKLEDNPVENEEASTHEAASFEDKPENLPELAESSLPAGPAPEEGEGGPEPQPSADQGSAELCGSQEVDQPTSGADTGTSDASGGSRRAVSETESLDQGSEPSQVSSETEPSDPARTESVSEASTRPEGESDLECADSSCNESVTTQLSSVDTRCSSLESARFPETPAFSSQEEEDGACAAEPTSSGPAEGSQESVCTAGSLPVVQVPSGEDEGPGAESATVPDQEELGEVWQRRGSLEGAAAAAESPPQEEGSAGEAQGTCEGATAQEEGATGGSQANGHQPLRSLPSVRQDVSRYQRVDEALPPNWEARIDSHGRIFYVDHVNRTTTWQRPTAPPAPQVLQRSNSIQQMEQLNRRYQSIRRTMTNERPEENTNAIDGAGEEADFHQASADFRRENILPHSTSRSRITLLLQSPPVKFLISPEFFTVLHSNPSAYRMFTNNTCLKHMITKVRRDTHHFERYQHNRDLVGFLNMFANKQLELPRGWEMKHDHQGKAFFVDHNSRTTTFIDPRLPLQSSRPTSALVHRQHLTRQRSHSAGEVGEDSRHAGPPVLPRPSSTFNTVSRPQYQDMVPVAYNDKIVAFLRQPNIFEILQERQPDLTRNHSLREKIQFIRTEGTPGLVRLSSDADLVMLLSLFEEEIMSYVPPHALLHPSYCQSPRGSPVSSPQNSPGTQRANARAPAPYKRDFEAKLRNFYRKLETKGYGQGPGKLKLIIRRDHLLEDAFNQIMGYSRKDLQRNKLYVTFVGEEGLDYSGPSREFFFLVSRELFNPYYGLFEYSANDTYTVQISPMSAFVDNHHEWFRFSGRILGLALIHQYLLDAFFTRPFYKALLRILCDLSDLEYLDEEFHQSLQWMKDNDIHDILDLTFTVNEEVFGQITERELKPGGANIPVTEKNKKEYIERMVKWRIERGVVQQTESLVRGFYEVVDARLVSVFDARELELVIAGTAEIDLSDWRNNTEYRGGYHDNHIVIRWFWAAVERFNNEQRLRLLQFVTGTSSIPYEGFASLRGSNGPRRFCVEKWGKITALPRAHTCFNRLDLPPYPSFSMLYEKLLTAVEETSTFGLE

SEQ ID NO: 56 - protein, 34 aa, Artificial Sequence
  Synthetic
EALPPNWEARIDSHGRIFYVDHVNRTTTWQRPTA

SEQ ID NO: 57 - protein, 34 aa, Artificial Sequence
  Synthetic
LELPRGWEMKHDHQGKAFFVDHNSRTTTFIDPRL

SEQ ID NO: 58 - protein, 390 aa, Homo sapiens
MAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRPISASYPPYQATGPPNTSYMPGMPGGISPYPSGYPPNPSGYPGCPYPPGGPYPATTSSQYPSQPPVTTVGPSRDGTISEDTIRASLISAVSDKLRWRMKEEMDRAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 59 - protein, 391 aa, Mus musculus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTATGPPNTSYMPGMPSGISAYPSGYPPNPSGYPGCPYPPAGPYPATTSSQYPSQPPVTTVGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 60 - protein, 391 aa, Rattus norvegicus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTAAGPPNTSYLPSMPSGISAYPSGYPPNPSGYPGCPYPPAGPYPATTSSQYPSQPPVTTAGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 61 - protein, 1493 aa, Artificial Sequence
  Synthetic
METDTLLLWVLLLWVPGSTGDMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTGGGPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGGGDYKDDDDK

SEQ ID NO: 62 - protein, 452 aa, Artificial Sequence
  Synthetic
METDTLLLWVLLLWVPGSTGDIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLGGGWYIWLGFIAGLIAIVMVTIMLGGGPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGGGDYKDDDDK

SEQ ID NO: 63 - protein, 324 aa, Artificial Sequence
  Synthetic
METDTLLLWVLLLWVPGSTGDMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFAGGGPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGGGDYKDDDDK

SEQ ID NO: 64 - protein, 257 aa, Artificial Sequence
  Synthetic
METDTLLLWVLLLWVPGSTGDMYSFVSEETGTLIVNSVLLFLAFVVFLLVTLAILTALGGGPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGGGDYKDDDDK

SEQ ID NO: 65 - protein, 8 aa, Artificial Sequence
  Synthetic
DYKDDDDK

SEQ ID NO: 66 - protein, 21 aa, Artificial Sequence
  Synthetic
METDTLLLWVLLLWVPGSTGD

SEQ ID NO: 67 - protein, 1273 aa, Artificial Sequence; identical to SEQ ID NO: 10
  Synthetic

SEQ ID NO: 68 - protein, 208 aa, Artificial Sequence
  Synthetic
IVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNL

SEQ ID NO: 69 - protein, 21 aa, Artificial Sequence
  Synthetic
WYIWLGFIAGLIAIVMVTIML

SEQ ID NO: 70 - protein, 104 aa, Artificial Sequence; identical to SEQ ID NO: 8
  Synthetic

SEQ ID NO: 71 - protein, 37 aa, Artificial Sequence
  Synthetic
MYSFVSEETGTLIVNSVLLFLAFVVFLLVTLAILTAL

SEQ ID NO: 72 - protein, 185 aa, Artificial Sequence
  Synthetic
PLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRT

SEQ ID NO: 73 - protein, 3 aa, Artificial Sequence
  Synthetic
GGG

(19) United States
(12) Patent Application Publication
(10) Pub. No.: US 2023/0391834 A1
(43) Pub. Date: Dec. 7, 2023

(54) WW-DOMAIN-ACTIVATED EXTRACELLULAR VESICLES TARGETING CORONAVIRUSES

(71) Applicant: President and Fellows of Harvard College, Cambridge, MA (US)

(72) Inventors: Quan Lu, Cambridge, MA (US); Sengjin Choi, Cambridge, MA (US)

(73) Assignee: President and Fellows of Harvard College, Cambridge, MA (US)

(21) Appl. No.: 18/032,134

(22) PCT Filed: Oct. 15, 2021
(86) PCT No.: PCT/US2021/055154

§ 371 Date: Apr. 14, 2023

Related U.S. Application Data
(60) Provisional application No. 63/093,107, filed on Oct. 16, 2020.

(51) Int. Cl. C07K 14/165; C12N 9/10; C07K 14/47

(52) U.S. Cl. CPC C07K 14/165; C12N 9/104; C07K 14/47; C07K 2319/95; C07K 2319/03

(57) ABSTRACT

Disclosed herein are methods, systems, compositions and strategies for the creation and use WW-domain-Activated Extracellular Vesicles, or WAEVs for presenting SARS-CoV-2 antigen domains, for example the SARS-CoV-2 M protein, the SARS-CoV-2 E protein, or the SARS-CoV-2 S protein. These WAEVs can be harnessed to deliver and present SARS-CoV-2 antigens useful for vaccine development.

### RELATED APPLICATIONS

This application claims priority under 35 U.S.C. § 119(e) to U.S. Provisional Application U.S. Ser. No. 63/093,107, filed Oct. 16, 2020, the contents of which are incorporated herein by reference.

### BACKGROUND OF THE INVENTION

The SARS-COV-2 virus, a coronavirus, has caused a global pandemic. SARS-COV-2 virus has several viral proteins on its surface, including the spike (S) protein, the membrane (M) protein, and the envelope (E) protein. Therefore, there is a need for new compositions and methods to present and deliver these viral surface proteins, as well as surface proteins of other coronaviruses, to elicit the production of neutralizing antibodies against coronaviruses such as SARS-COV-2 as a new strategy to develop effective coronavirus vaccines.

### SUMMARY OF THE INVENTION

The present disclosure relates, at least in part, to novel extracellular vesicles (EVs) which contain WW-domain containing proteins with extracellular domains (WW-domain-Activated Extracellular Vesicles, or WAEVs) directed to antigens for coronaviruses, including the spike (S) protein, the membrane (M) protein, and the envelope (E) protein of SARS-COV-2. The S, M, and E proteins can be displayed on the surface of novel EVs through the introduction of WW-domain containing proteins that are fused to a transmembrane domain associated (e.g., covalently linked) with these surface proteins.

Direct fusions of transmembrane-containing proteins to arrestin domain containing protein 1 (ARDDC1) result in decreased or abolished budding activity of ARRCC1. WAEVs are able to bud independent of ARRDC1, and do not appear to be enhanced by ARDDC1 overexpression. In addition, WAEVs do not appear to be like classical exosomes because they do not contain one or more of the typical exosomal markers (e.g., CD63; CD81, CD9, and PTGFRN). Instead, other proteins may be responsible for mediating WAEV budding, including the secretory carrier-associated membrane protein 3 (SCAMP3). WAEVS can be used to deliver and present viral antigens useful for vaccine development, such as coronavirus antigens (including the S, M, and E proteins of SARS-COV-2).

Accordingly, in some aspects, the disclosure relates to a fusion protein comprising: (a) a WW-containing domain; (b) a transmembrane domain; and (c) an extracellular domain, wherein the extracellular domain comprises a coronavirus antigen domain. In some embodiments, the coronavirus antigen domain is a SARS-COV-2 antigen domain.

In some embodiments, the WW-containing domain of any of the fusion proteins of the disclosure comprise at least one WW domain. In some embodiments, the WW-containing domain of any of the fusion proteins of the disclosure comprise at least two WW domain. In some embodiments, the WW-containing domain of any of the fusion proteins of the disclosure comprise at least three WW domain. In some embodiments, the WW-containing domain of any of the fusion proteins of the disclosure comprise at least four WW domain. In some embodiments, the fusion protein comprises at least one WW domain which is an ITCH protein WW domain. In some embodiments, the WW-containing domain of any of the fusion proteins of the disclosure comprise a sequence having at least 95% identity to the sequence of SEQ ID NO: 1. In some embodiments, the WW-containing domain of any of the fusion proteins of the disclosure comprise the sequence of SEQ ID NO: 1.

In some embodiments, the transmembrane domain of any of the fusion proteins of the disclosure comprises a coronavirus transmembrane domain. In some embodiments, the coronavirus transmembrane domain is a SARS-COV-2 transmembrane domain. In some embodiments, the transmembrane domain of any of the fusion proteins of the disclosure comprises a SARS-COV-2 M protein transmembrane domain, a SARS-COV-2 E protein transmembrane domain, or a SARS-COV-2 S protein transmembrane domain. In some embodiments, the transmembrane domain comprises a sequence having at least 95% identity to the sequence of SEQ ID NO: 7, the sequence of SEQ ID NO: 9, or the sequence of SEQ ID NO: 10. In some embodiments, the transmembrane domain comprises the sequence of SEQ ID NO: 7, the sequence of SEQ ID NO: 9, or the sequence of SEQ ID NO:10.

In some embodiments, the extracellular domain of any of the fusion proteins of the disclosure comprises a coronavirus antigen domain. In some embodiments, the coronavirus antigen domain is a SARS-CoV-2 antigen domain. In some embodiments, the SARS-CoV-2 antigen domain is an M extracellular domain, an E protein extracellular domain, or a S protein extracellular domain. In some embodiments, the extracellular domain comprises a sequence having at least 95% identity to the sequence of SEQ ID NO: 7, the sequence of SEQ ID NO: 9., or the sequence of SEQ ID NO: 10. In some embodiments, the extracellular domain comprises the sequence of SEQ ID NO: 7, the sequence of SEQ ID NO: 9., the sequence of SEQ ID NO: 10.

In some embodiments, the fusion proteins of the disclosure further comprise a signal peptide.

In some aspects, the disclosure relates to an isolated nucleic acid encoding at least one of any of the fusion proteins of the disclosure.

In some embodiments, any of the isolated nucleic acids of the disclosure are operably linked to a promoter. In some embodiments, the promoter is a constitutive promoter, an inducible promoter, or a tissue specific promoter.

In some embodiments, any of the isolated nucleic acids of the disclosure comprise at least one additional regulatory sequence.

In some aspects, the disclosure relates to a WW protein domain activated extracellular vesicle (WAEV), comprising: (a) a lipid bilayer; and (b) a fusion protein as described herein.

In some embodiments, a WAEV as described herein, further comprises SCAMP3.

In some embodiments, a WAEV as described herein does not comprise at least one of the following exosomal markers: CD63; CD81, CD9, and/or PTGFRN.

In some aspects, the disclosure relates to a WAEV-producing cell, comprising: (a) a recombinant expression construct encoding at least one of any of the fusion proteins of the disclosure under the control of a heterologous promoter.

In some aspects, the disclosure relates to a WAEV-producing cell, comprising: (a) at least one of any of the isolated nucleic acids of the disclosure.

In some aspects, the disclosure relates to a method of delivering WAEVs displaying a COVID-19 antigenic peptide, comprising: delivering at least one of any of the fusion proteins of the disclosure, at least one of any of the isolated nucleic acids of the disclosure, at least one of any of the WAEVs of the disclosure, and/or at least one of any of the WAEV-producing cells of the disclosure, wherein the extracellular protein of the fusion protein comprises a COVID-19 antigenic peptide.

In some embodiments, the subject is mammalian. In some embodiments, the subject is human.

Other advantages, features, and uses of the invention will be apparent from the detailed description of certain exemplary, non-limiting embodiments; the drawings; the non-limiting working examples; and the claims.

### BRIEF DESCRIPTION OF THE DRAWINGS

FIG. 1 shows the human ITCH protein sequence with its 4 WW-domains highlighted. Underlined sequences are used to make 4WW-fusion constructs.

FIGS. 2A-2B show that SCAMP3 has the elements necessary to drive the formation of WAEVs. FIG. 2A shows SCAMP3 protein contains both PPXY (SEQ ID NO: 22) and PSAP (SEQ ID NO: 17) motifs, which can interact with WW domains and TSG101, respectively. Also highlighted are the four transmembrane domains. FIG. 2B shows a model in which SCAMP3, which sits on the plasma membrane, recruits TSG101 and WW domain-linked protein cargo (with its own or engineered transmembrane domain [TM]) to drive the formation of WAEVs.

FIG. 3 shows a schematic drawing of several viral proteins on the surface of a SARS-CoV-2 virus. The viral proteins include the spike (S) protein, the membrane (M) protein, and the envelope (E) protein.

FIGS. 4A-4B show SARS-CoV-2 viral protein-4WW fusion constructs. FIG. 4A shows schematic representation of SARS-CoV-2 viral protein-4WW fusion constructs. RBD: receptor binding domain of spike protein. M: membrane protein. TM: transmembrane domain; ED: extracellular domain; SP: Signal peptide (Igk leader). FIG. 4B shows a schematic representation of extracellular vesicles (EV) with SARS-COV-2 membrane (M) protein presented on the surface.

FIGS. 5A-5B show SARS-CoV-2 viral protein-4WW fusion constructs. FIG. 5A shows a schematic representation of extracellular vesicles (EV) with SARS-CoV-2 envelope (E) protein presented on the surface. FIG. 5B shows a schematic representation of the E-4WW fusion construct. SP: Signal peptide (Igk leader).

FIGS. 6A-6B show SARS-CoV-2 viral protein-4WW fusion constructs. FIG. 6A shows a schematic representation of extracellular vesicles (EV) with SARS-CoV-2 spike (S) protein presented on the surface. FIG. 6B shows a schematic representation of the S-4WW fusion construct. SP: Signal peptide (Igk leader).

FIGS. 7A-7C show the budding of SARS-CoV-2 WAEVs. FIG. 7A shows the expression and budding of the M-4WW fusion protein into EVs in HEK293T cells. Indicated fusion or control constructs were transfected into HEK293T (WT) or HEK29T ARRDC1-knockout (ARRDC1-KO) cells. 48 hours post transfection, EVs were isolated via ultracentrifugation. Western blotting was done on the EVs along with whole cell lysates with indicated antibodies. FIG. 7B shows the budding of the M-4WW fusion protein into EVs in HEK293T cells. Indicated fusion or control constructs were transfected into HEK29T ARRDC1-knockout (ARRDC1-KO) cells. 48 hours post transfection, EVs were isolated via ultracentrifugation. Western blotting was done on the EVs along with whole cell lysates with indicated antibodies. FIG. 7C shows the budding of the E-4WW fusion protein into EVs. Indicated fusion or control constructs were transfected into HEK29T ARRDC1-knockout (ARRDC1-KO) cells. 48 hours post transfection, EVs were isolated via ultracentrifugation. Western blotting was done on the EVs along with whole cell lysates with indicated antibodies.

FIGS. 8A-8B show the characterization and purification of SARS-CoV-2 M-4WW WAEVs. FIG. 8A shows a Western blot analysis of M-4WW EV after Optiprep density gradient purification. Western blotting for FLAG, CD9, Vinculin were done on both the whole cell lysate and EV. FIG. 8B shows a Western blot analysis of M-4WW EV after Optiprep density gradient purification. Western blotting for FLAG, CD9 were done on both the whole cell lysate and EV.

FIG. 9 shows the size distribution of M-4WW EV. EVs from HEK293T-ARRDC1-KO cells transfected with M-4WW construct were analyzed from 5 independent experiments using the NanoSight particle analysis system (NS300). Data are presented as the mean.

### DEFINITIONS

### Antigen

The term “antigen,” as may be used herein refers to a molecule that provokes an immune response. This immune response may involve either antibody production, or the activation of specific immunologically competent cells, or both. The skilled artisan will understand and readily appreciate that any macromolecule, including virtually all proteins or peptides, can serve as an antigen. Furthermore, antigens can be derived from recombinant or genomic nucleic acid. A skilled artisan will understand that any nucleic acid, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated and/or synthesized or can be derived from a biological sample. Such a biological sample can include, but is not necessarily limited to a tissue sample, a tumor sample, a cell, or a biological fluid. In some embodiments, the antigen is a protein, or fragment thereof. In some embodiments, the antigen is a nucleic acid, or fragment thereof. In some embodiments, the WAEVs of the present disclosure comprise an antigen as an extracellular domain or part of an extracellular domain. In some embodiments, the WAEVs of the present disclosure present an antigen on the membrane of the WAEV. In some embodiments, the fusion proteins of the present disclosure comprise an antigen as an extracellular domain or part of an extracellular domain.

Associated With

The term “associated with,” as may be used herein, refers to a property of two or more entities, for example, chemical moieties, molecules (e.g., domains, nucleic acids, peptides), and/or WAEVs, and means that the entities are physically in contact or connected with one another, either directly or via one or more additional moieties that serves as a linker, to form a structure that is sufficiently stable so that the entities remain physically in contact under the conditions in which the structure is used, e.g., physiological conditions. A WAEV can be associated with an agent, for example, a nucleic acid, protein, or small molecule, by a mechanism that involves a covalent or non-covalent association. For example, a WW-domain containing fusion protein of the present invention can be associated with a protein containing PPXY (SEQ ID NO: 22) motifs, such as a NEDD4 E3 ligase proteins, including but not limited to SCAMP3. In certain embodiments, the agent to be delivered (e.g., an extracellular domain cargo protein, which can be or can include an antigen) is covalently bound (e.g. fused) to a transmembrane domain and a WW-containing domain, and this fusion protein can be non-covalently bound to a protein containing PPXY (SEQ ID NO: 22) motif, including but not limited to a SCAMP3 protein or variant thereof. In some embodiments, an association is via a linker, which can be, but is not limited to, a nucleic acid or amino acid linker, for example, a cleavable linker.

### Cargo

The term “cargo,” as may be used herein, refers to an antigen, protein, or peptide that may be incorporated in a WAEV, for example, as an extracellular domain of the WAEV. The term “delivered” as it relates to cargo refers to any antigen, protein, or peptide that can be delivered via its association with or inclusion in a WAEV to a subject, organ, tissue, or cell. In some embodiments, the cargo is to be delivered to a target cell in vitro, in vivo, or ex vivo. In some embodiments, the cargo to be delivered is an antigen that is presented on the surface of a WAEV.

In general, a “small molecule” refers to a substantially non-peptide, non-oligomeric organic compound either prepared in the laboratory or found in nature. Small molecules, as used herein, can refer to compounds that are “natural product-like,” however, the term “small molecule” is not limited to “natural product-like” compounds. Rather, a small molecule is typically characterized in that it contains several carbon-carbon bonds, and has a molecular weight of less than 2000 g/mol, less than 1500 g/mol, less than 1250 g/mol, less than 1000 g/mol, less than 750 g/mol, less than 500 g/mol, or less than 250 g/mol, although this characterization is not intended to be limiting for the purposes of the present invention. In certain other embodiments, natural-product-like small molecules are utilized.

### Effective Amount

The term “effective amount” refers to an amount of a composition (e.g., a WAEV as described herein) sufficient to elicit a desired biological response. For example, in some embodiments, an effective amount of a WAEV as described herein may refer to the amount of the WAEV as described herein sufficient to elicit an immune reaction to the extracellular domain contained (e.g., presented) therein, or thereon (e.g., antigen, or fragment thereof). As will be appreciated by the skilled artisan, the effective amount of a composition (e.g., a WAEV) as described herein may vary depending on various factors as, for example, the desired biological response, the cell or tissue being targeted, and/or the agent being used.

### Extracellular Domain

The terms “extracellular domain” and “exterior domain,” as may be used interchangeably herein, refer to the domain of an antigen, protein, or peptide which is present on the exterior of a membrane of a membrane-containing molecule (e.g., cell, vesicle, EV, and/or WAEV). In some embodiments, the extracellular domain comprises a domain of a fusion protein. The extracellular domain may be the terminal domain of a protein. In some embodiments, the extracellular domain is associated to the transmembrane domain by one terminus. In some embodiments, the extracellular domain is associated to the transmembrane domain through its N terminus (e.g., directly or indirectly). In some embodiments, the extracellular domain is associated to the transmembrane domain through its C terminus (e.g., directly or indirectly). In some embodiments, extracellular domain is linked or fused directly to the transmembrane domain. In some embodiments, the extracellular domain is linked indirectly to the transmembrane domain, for example through a linker. In some embodiments, the extracellular domain is indirectly linked to the transmembrane domain through another protein domain. In some embodiments, the extracellular domain is indirectly linked to the transmembrane domain through a linker.

In some embodiments, the extracellular domain is positioned such that all of the extracellular domain is exterior of a membrane to which it is associated. It should be noted that while the term “extracellular” can be used in the context of the membrane of a cell, as used herein, the term shall not solely refer to such context, and shall also refer to domains which are associated with a membrane as described herein which may not be a cell, for example, without limitation, an extracellular vesicle such as a WAEV. In some embodiments, only a portion of the extracellular domain is exterior to a membrane to which it is associated. In some embodiments, the membrane is a lipid-based layer. In some embodiments, the lipid-based layer is a lipid bilayer. In some embodiments, the lipid membrane is a cellular membrane. In some embodiments, the lipid membrane is a lipid layer of an extracellular vesicle. In some embodiments, the extracellular vesicle is a WAEV.

Any extracellular domain is contemplated for use herein. In some embodiments, the extracellular domain is or comprises an extracellular domain of a known protein. In some embodiments, the extracellular domain is or comprises a fragment of a known protein. In some embodiments, the extracellular domain is or comprises an antigen domain, or fragment thereof. In some embodiments, the extracellular domain is or comprises a viral protein, or fragment thereof. In some embodiments, the extracellular domain is or comprises a viral antigen protein or viral antigen domain, or fragment thereof. In some embodiments, the viral antigen domain is a coronavirus domain, including but not limited to a SARS-CoV-2-derived protein or variant thereof. For example, in some embodiments, the extracellular domain is or comprises the SARS-CoV-2 M protein, SARS-CoV-2 E protein, or SARS-CoV-2 S protein. Extracellular domain can be identified using any method known in the art or described herein, e.g., by using the UniProt Database.

### Fusion Protein

The term “fusion protein,” as may be used herein, refers to a hybrid (e.g., chimeric, recombinant) polypeptide which comprises protein domains from at least two different proteins. One protein domain may be located at the amino-terminal (N-terminal) portion of the fusion protein and will contain the free N terminus (e.g., amino (NH₂) group) of the fusion protein, and this protein domain of the fusion protein may be referred to as the “amino-terminal fusion protein” or “amino-terminal fusion protein domain.” Similarly, one protein domain may be located at the carboxy-terminal (C-terminal) portion of the fusion protein and will contain the free C terminus (e.g., carboxyl (COOH) group) of the fusion protein, this protein domain of the fusion protein may be referred to as the “carboxy-terminal fusion protein” or “carboxy-terminal fusion protein domain.” In some embodiments, fusion proteins may comprise additional protein domains. In some embodiments, the additional protein domains may be similar or distinct from the amino-terminal fusion protein domain and/or carboxy-terminal fusion protein domain. These additional domains will be positioned between the amino-terminal fusion protein domain and the carboxy-terminal fusion protein domain. In some embodiments, a protein domain of a fusion protein may comprise a WW-containing domain. In some embodiments, a protein domain of a fusion protein may comprise a transmembrane domain. In some embodiments, a protein domain of a fusion protein may comprise an extracellular domain. Any of the fusion proteins provided herein may be produced by any method known in the art. For example, the proteins provided herein may be produced via recombinant protein expression and purification, which is especially suited for fusion proteins comprising a peptide linker. Methods for fusion protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference. A fusion protein can be encoded by a recombinant nucleic acid (e.g., DNA, RNA).

### Isolated

The term “isolated,” as may be used herein, refers to a characteristic of a material as provided herein (e.g., nucleic acid (e.g., RNA, DNA, polynucleotide), amino acid, peptide (e.g., polypeptide, protein), vector (e.g., viral vector (e.g., adeno-associated viral vector))), as being altered or removed from its natural state (i.e., native or original environment if it is naturally occurring) where such material would otherwise be found. Therefore, a naturally occurring nucleic acid or peptide present in a living animal is not isolated, but the same nucleic acid or peptide, separated by human intervention from some or all of the coexisting materials in the natural system, is “isolated.” For example, a nucleic acid or a peptide naturally present in a living animal is not “isolated,” but the same nucleic acid or peptide partially or completely separated from the coexisting materials of its natural state or host is “isolated.” An artificial, recombinant, or engineered material, for example, a non-naturally occurring nucleic acid construct or peptide construct, are, accordingly, also referred to as isolated. An isolated material can exist in substantially purified form, or can exist in a non-native environment such as, for example, a vector or host cell, however, a material does not have to be purified in order to be isolated. Accordingly, a material may be part of a vector and/or part of a composition, and still be isolated in that such vector or composition is not part of the environment in which the material is found in its natural state.

### Linker

The term “linker,” as may be used herein, refers to a chemical moiety linking two molecules or moieties, e.g., a WW-containing domain, transmembrane domain, extracellular domain, and/or any other molecule (e.g., peptide, tag, nucleic acid). Typically, the linker is positioned between, or flanked by, two groups, molecules, or other moieties and connected to each one via a covalent bond, thus connecting the two. In some embodiments, the linker comprises an amino acid or a plurality of amino acids (e.g., a peptide or protein). In some embodiments, the linker comprises a nucleotide (e.g., DNA or RNA) or a plurality of nucleotides (e.g., a nucleic acid). In some embodiments, the linker is an organic molecule, functional group, polymer, or other chemical moiety. In some embodiments, the linker is a cleavable linker, e.g., the linker comprises a bond that can be cleaved upon exposure to, for example, UV light or a hydrolytic enzyme, such as a protease or esterase. In some embodiments, the linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids). In other embodiments, the linker is a chemical bond (e.g., a covalent bond, amide bond, disulfide bond, ester bond, carbon-carbon bond, carbon heteroatom bond).

### Nucleic Acid

The terms “nucleic acid,” “nucleotide sequence,” “polynucleotide,” “oligonucleotide,” and “polymer of nucleotides” as may be used interchangeably herein, refer to a string of at least two, base-sugar-phosphate combinations and includes, among others, single-stranded and double-stranded DNA, DNA that is a mixture of single-stranded and double-stranded regions, single-stranded and double-stranded RNA, and RNA that is mixture of single-stranded and double-stranded regions, hybrid molecules comprising DNA and RNA that may be single stranded or, more typically, double stranded or a mixture of single-stranded and double-stranded regions. In addition, the terms (e.g., nucleic acid, et al.) as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often referred to as an oligonucleotide.

The terms (e.g., nucleic acid, et al.) also encompass such chemically, enzymatically, or metabolically modified forms of nucleic acids, as well as the chemical forms of DNA and RNA characteristic of viruses and cells, including simple and complex cells. For instance, the terms (e.g., nucleic acid, et al.) as used herein can include DNA or RNA as described herein that contain one or more modified bases. The nucleic acids may also include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, O(6) methylguanine, 4-acetylcytidine, (carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages). Thus, DNA or RNA including unusual bases, such as inosine, or modified bases, such as tritylated bases, to name just two examples, are nucleic acids as the term is used herein. The terms (e.g., nucleic acid, et al.) also includes peptide nucleic acids (PNAs), phosphorothioates, and other variants of the phosphate backbone of native nucleic acids. Natural nucleic acids have a phosphate backbone, artificial nucleic acids can contain other types of backbones, but contain the same bases. Thus, DNA or RNA with backbones modified for stability or for other reasons are nucleic acids as that term is intended herein.

### Operably Linked

The term “operably linked,” as may be used herein, refers to an arrangement of sequences or regions wherein the components are configured so as to perform their usual or intended function. Thus, a regulatory or control sequence operably linked to a coding sequence is capable of affecting the expression of the coding sequence. The regulatory or control sequences need not be contiguous with the coding sequence, so long as they function to direct the proper expression or polypeptide production. Thus, as a non-limiting example, intervening untranslated but transcribed sequences can be present between a promoter sequence and the coding sequence and the promoter sequence can still be considered operably linked to the coding sequence. A promoter sequence, as described herein, is a DNA regulatory region a short distance from the 5′ end of a gene that acts as the binding site for RNA polymerase. The promoter sequence may bind RNA polymerase in a cell and/or initiate transcription of a downstream (3′ direction) coding sequence. The promoter sequence may be a promoter capable of initiating transcription in prokaryotes or eukaryotes. Some non-limiting examples of eukaryotic promoters include the cytomegalovirus (CMV) promoter, the chicken beta-actin (β-actin) (CBA) promoter, and a hybrid form of the CBA promoter (CBh).

### Percent Identity

The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category.

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%,at least 96%, at least 96.5%,at least 97%, at least 97.5%,at least 98%, at least 98.5%,at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.).

### Regulatory Sequence

The terms “regulatory sequence,” “regulatory signal,” “control sequence,” and “control signal,” as may be used interchangeably herein, refer to sequences that are responsible for expressing a particular nucleic acid or may include other sequences, such as heterologous, synthetic, or partially synthetic sequences. The sequences can be of eukaryotic, prokaryotic, or viral origin that stimulate or repress transcription of a gene in a specific or non-specific manner and in an inducible or non-inducible manner. Regulatory or control regions may include origins of replication, RNA splice sites, introns, chimeric or hybrid introns, promoters, enhancers, transcriptional termination sequences, poly A sites, locus control regions, signal sequences that direct the polypeptide into the secretory pathways of the target cell, and introns. A heterologous regulatory region is not naturally associated with the expressed nucleic acid to which it is linked. Included among the heterologous regulatory regions are regulatory regions from a different species, regulatory regions from a different gene, hybrid regulatory sequences, and regulatory sequences that do not occur in nature, but which are designed by one of ordinary skill in the art.

### Reporter

The terms “reporter,” “reporter tag,” “signal,” and “signal tag,” as such terms may be used interchangeably herein, refer a molecule (e.g., peptide, nucleic acid, other moiety) which is associated with a subject molecule to identify the subject molecule during use (e.g., in vivo, in vitro, ex vivo). Any suitable reporter is contemplated for use herein. Reporter and signals are well known in the art and the selection and use of such reporters will be readily appreciated by the skilled artisan. For example, without limitation, green fluorescent protein is a protein isolated from the jellyfish Aequorea victoria that fluoresces green when exposed to blue light (e.g., an enhanced or wavelength-shifted version of the protein). In some embodiments, a reporter or signal is green fluorescent protein (GFP).

### Subject

The term “subject,” as used herein, refers to any organism in need of the use of the subject matter herein. In some embodiments, the use includes treatment using, or diagnosis using, the subject matter herein. For example, without limitation, subjects may include mammals and non-mammals. As used herein, a “mammal,” refers to any animal constituting the class Mammalia (e.g., a human, mouse, rat, cat, dog, sheep, rabbit, horse, cow, goat, pig, guinea pig, hamster, chicken, turkey, or a non-human primate (e.g., Marmoset, Macaque)). In some embodiments, the mammal is a human.

### Target Cell

The term “target cell” as used herein, refers to a cell which is the intended or desired target of the intervention, action, or effect which is intended or desired by the intervention of a method or composition. In some embodiments, the target cell is a cell that can host, replicate, and express an isolated nucleic acid, fusion protein, microvesicle, or WAEV as described herein. In some embodiments, the target cell is the cell to which the delivery of a therapeutic molecule is directed, for example, such as when a WAEV displays a homing molecule for such a target cell. In some embodiments, a host cell is taken from a subject. In some embodiments, the host cell is derived from cells not taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Pancl, PC-3, TF1, CTLL-2, C1R, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr -/-, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HEK293T, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (e.g., the American Type Culture Collection (ATCC) (Manassus, Va.)).

### Transmembrane Domain

The term “transmembrane domain,” as may be used herein, refers to the domain of a protein or polypeptide which spans the membrane of a membrane-contained molecule (e.g., cell, vesicle, EV, or WAEV), potentially associating multiple domains of a larger protein structure (e.g., WW-containing domain, extracellular domain). In some embodiments, the transmembrane domain comprises a domain of a fusion protein. In some embodiments, the transmembrane domain is positioned centrally to a domain located interior of a membrane and a domain exterior to a membrane. In some embodiments, the membrane is a lipid-based layer. In some embodiments, the lipid-based layer is a lipid bilayer. In some embodiments, the lipid layer is a lipid monolayer. In some embodiments, the lipid membrane is a cellular membrane. In some embodiments, the lipid membrane is a lipid layer of an extracellular vesicle. In some embodiments, the extracellular vesicle is a WAEV. The transmembrane domain may span the membrane one time or multiple times and can be responsible for connecting the domains of the fusion protein across the membrane. Any transmembrane domain is contemplated for use herein. Transmembrane domains can be identified using any method known in the art or described herein, e.g., by using the UniProt Database.

### Treatment

The terms “treatment,” “treat,” and “treating,” as may be used interchangeably herein, refer to partially or completely alleviating, ameliorating, relieving, delaying onset of, inhibiting progression of, reducing severity of, and/or reducing incidence of one or more symptoms or features of a particular indication, disease, disorder, condition, and/or symptom thereof. In some embodiments, the treatment refers to a clinical intervention. In some embodiments, treatment may be administered after one or more symptoms have developed and/or after a disease has been diagnosed. In other embodiments, treatment may be administered in the absence of symptoms (e.g., to prevent or delay onset of a symptom or inhibit onset or progression of a disease). For example, treatment may be administered to a susceptible individual (e.g., subject) prior to the onset of symptoms (e.g., in light of a history of symptoms and/or in light of genetic or other susceptibility factors). In some embodiments, the treatment is used and/or administered as a prophylaxis. Treatment may also be continued after symptoms have resolved, for example, to prevent or delay their recurrence.

WW-Containing Domain

The terms “WW-containing domain” and “WW domain” as may be used interchangeably herein, refer to a protein domain having two basic residues at the C terminus that mediates protein-protein interactions with short proline-rich or proline-containing motifs. It should be appreciated that the two basic residues (e.g., any two of: histidine (H), arginine (R), and/or lysine (K)) of the WW-containing domain are not required to be at the absolute C terminus of the WW-containing protein domain (e.g., the final residues of the C terminus). Rather, the two basic residues may be at a C-terminal portion of the WW-containing protein domain (e.g., the C-terminal half of the WW-containing protein domain). In some embodiments, the WW-containing domain contains at least 1, 2, 3, 4, 5, 6, 7, 8, 9, or 10 tryptophan (W) residues. In some embodiments, the WW-containing domain contains at least two W residues. In some embodiments, the at least two W residues are spaced apart by from 15-25 amino acids. In some embodiments, the at least two W residues are spaced apart by from 19-23 amino acids. In some embodiments, the at least two W residues are spaced apart by from 20-22 amino acids. The WW-containing domain possessing the two basic C-terminal amino acid residues may have the ability to associate with short proline-rich or proline-containing motifs (e.g., a PPXY (SEQ ID NO: 22) motif). WW-containing domains bind a variety of distinct peptide ligands including motifs with core proline-rich sequences, such as PPXY (SEQ ID NO: 22), such as is found in SCAMPS (among others). A WW-containing domain may be a 30-40 amino acid protein interaction domain with two signature tryptophan residues spaced by 20-22 amino acids. The three-dimensional structure of WW-containing domains shows that they generally fold into a three-stranded, antiparallel β sheet with two ligand-binding grooves.

WW-containing domains are found in many eukaryotes and are present in approximately 50 human proteins (Bork, P. & Sudol, M. The WW domain: a signaling site in dystrophin? Trends Biochem Sci 19, 531-533 (1994)). WW-containing domains may be present together with several other interaction domains, including membrane targeting domains, such as C2 in the NEDD4 family proteins, the phosphotyrosine-binding (PTB) domain in FE65 protein, FF domains in CA150 and FBPI1, and pleckstrin homology (PH) domains in PLEKHA5. The NEDD4 E3 ligase proteins include, but are not necessarily limited to, ITCH, NEDD4, NEDD4 L, WWP1, WWP2, Smurf1, Smurf2, BUL1, and NEDL2. WW-containing domains are also linked to a variety of catalytic domains, including HECT E3 protein-ubiquitin ligase domains in NEDD4 family proteins, rotomerase or peptidyl prolyisomerase domains in Pinl, and Rho GAP domains in ArhGAP9 and ArhGAP12.

In the instant disclosure, the WW-containing domain may be a WW-containing domain that naturally possesses two basic amino acids at the C terminus. In some embodiments, a WW-containing domain or WW-containing domain variant may be from the human ubiquitin ligase WWP1, WWP2, Nedd4-1, Nedd4-2, Smurf1, Smurf2, ITCH, NEDL1, or NEDL2. Exemplary amino acid sequences of WW-containing domain containing proteins (WW-containing domains underlined) are listed below. It should be appreciated that any of the WW-containing domains or WW-containing domain variants of the exemplary proteins may be used in the invention, described herein, and are not meant to be limiting.

Human WWP1 amino acid sequence (uniprot.org/uniprot/Q9HOM0). The four underlined WW domains correspond to amino acids 349-382 (WW1), 381-414 (WW2), 456-489 (WW3), and 496-529 (WW4).

| (SEQ ID NO: 25) | |
|---|---|
| MATASPRSDT SNNHSGRLQL QVTVSSAKLK RKKNWFGTAI YTEVVVDGEI | 50 |
| TKTAKSSSSS NPKWDEQLTV NVTPQTTLEF QVWSHRTLKA DALLGKATID | 100 |
| LKQALLIHNR KLERVKEQLK LSLENKNGIA QTGELTVVLD GLVIEQENIT | 150 |
| NCSSSPTIEI QENGDALHEN GEPSARTTAR LAVEGINGID NHVPTSTLVQ | 200 |
| NSCCSYVVNG DNTPSSPSQV AARPKNTPAP KPLASEPADD TVNGESSSFA | 250 |
| PTDNASVIGT PVVSEENALS PNCTSTTVED PPVQEILTSS ENNECIPSTS | 300 |
| AELESEARSI LEPDTSNSRS SSAFEAAKSR QPDGCMDPVR QQSGNANTET | 350 |
| LPSGWEQRKD PHGRTYYVDH NTRTTTWERP QPLPPGWERR VDDRRRVYYV | 400 |
| DHNTRTTTWQ RPTMESVRNF EQWQSQRNQL QGAMOQFNQR YLYSASMLAA | 450 |
| ENDPYGPLPP GWEKRVDSTD RVYFVNHNTK TTQWEDPRTQ GLQNEEPLPE | 500 |
| GWEIRYTREG VRYFVDHNTR TTTFKDPRNG KSSVTKGGPQ IAYERGFRWK | 550 |
| LAHFRYLCQS NALPSHVKIN VSRQTLFEDS FQQIMALKPY DLRRRLYVIF | 600 |
| RGEEGLDYGG LAREWFFLLS HEVLNPMYCL FEYAGKNNYC LQINPASTIN | 650 |
| PDHLSYFCFI GRFIAMALFH GKFIDIGFSL PFYKRMLSKK LTIKDLESID | 700 |
| TEFYNSLIWI RDNNIEECGL EMYFSVDMEI LGKVTSHDLK LGGSNILVTE | 750 |
| ENKDEYIGLM TEWRFSRGVQ EQTKAFLDGF NEVVPLQWLQ YFDEKELEVM | 800 |
| LCGMQEVDLA DWQRNTVYRH YIRNSKQIIW FWQFVKETDN EVRMRLLQFV | 850 |
| TGTCRLPLGG FAELMGSNGP QKFCIEKVGK DTWLPRSHTC FNRLDLPPYK | 900 |
| SYEQLKEKLL FAIEETEGFG QE | 922 |

WW1 (349-382):
(SEQ ID NO: 26)
ETLPSGWEQRKDPHGRTYYVDHNTRTTTWERPQP.
WW2 (381-414):
(SEQ ID NO: 27)
QPLPPGWERRVDDRRRVYYVDHNTRTTTWQRPTM.
WW3 (456-489):
(SEQ ID NO: 28)
ENDPYGPLPPGWEKRVDSTDRVYFVNHNTKTTQWEDPRT.
WW4 (496-529):
(SEQ ID NO: 29)
EPLPEGWEIRYTREGVRYFVDHNTRTTTFKDPRN.

Human WWP2 amino acid sequence (uniprot.org/uniprot/ 000308). The four underlined WW domains correspond to amino acids 300-333 (WW1), 330-363 (WW2), 405-437 (WW3), and 444-547 (WW4).

| (SEQ ID NO: 30) | |
|---|---|
| MASASSSRAG VALPFEKSQL TLKVVSAKPK VHNRQPRINS YVEVAVDGLP | 50 |
| SETKKTGKRI GSSELLWNEI IILNVTAQSH LDLKVWSCHT LRNELLGTAS | 100 |
| VNLSNVLKNN GGKMENMQLT LNLQTENKGS VVSGGELTIF LDGPTVDLGN | 150 |
| VPNGSALTDG SQLPSRDSSG TAVAPENRHQ PPSTNCFGGR SRTHRHSGAS | 200 |
| ARTTPATGEQ SPGARSRHRQ PVKNSGHSGL ANGTVNDEPT TATDPEEPSV | 250 |
| VGVTSPPAAP LSVTPNPNTT SLPAPATPAE GEEPSTSGTQ QLPAAAQAPD | 300 |
| ALPAGWEQRE LPNGRVYYVD HNTKTTTWER PLPPGWEKRT DPRGRFYYVD | 350 |
| HNTRTTTWQR PTAEYVRNYE QWQSQRNQLQ GAMQHFSQRF LYQSSSASTD | 400 |
| HDPLGPLPPG WEKRQDNGRV YYVNHNTRTT QWEDPRTQGM IQEPALPPGW | 450 |
| EMKYTSEGVR YFVDHNTRTT TFKDPRPGFE SGTKQGSPGA YDRSFRWKYH | 500 |
| QFRFLCHSNA LPSHVKISVS RQTLFEDSFQ QIMNMKPYDL RRRLYIIMRG | 550 |
| EEGLDYGGIA REWFELLSHE VLNPMYCLFE YAGKNNYCLQ INPASSINPD | 600 |
| HLTYFRFIGR FIAMALYHGK FIDTGFTLPF YKRMLNKRPT LKDLESIDPE | 650 |
| FYNSIVWIKE NNLEECGLEL YFIQDMEILG KVTTHELKEG GESIRVTEEN | 700 |
| KEEYIMLLTD WRFTRGVEEQ TKAFLDGENE VAPLEWLRYF DEKELELMLC | 750 |
| GMQEIDMSDW QKSTIYRHYT KNSKQIQWFW QVVKEMDNEK RIRLLQFVTG | 800 |
| TCRLPVGGFA ELIGSNGPQK FCIDKVGKET WLPRSHTCEN RLDLPPYKSY | 850 |
| EQLREKLLYA IEETEGFGQE | 870 |

WW1 (300-333):
(SEQ ID NO: 31)
DALPAGWEQRELPNGRVYYVDHNTKTTTWERPLP.
WW2 (330-363):
(SEQ ID NO: 32)
PLPPGWEKRT DPRGRFYYVDHNTRTTTWQRPTA.
WW3 (405-437):
(SEQ ID NO: 33)
HDPLGPLPPGWEKRQDNGRVYYVNHNTRTTQWEDPRT.
WW4 (444-477):
(SEQ ID NO: 34)
PALPPGWEMKYTSEGVRYFVDHNTRTTTFKDPRP.

Human Nedd4-1 amino acid sequence (uniprot.org/uniprot/P46934). The four underlined WW domains correspond to amino acids 610-643 (WW1), 767-800 (WW2), 840-873 (WW3), and 892-925 (WW4).

| (SEQ ID NO: 35) | |
|---|---|
| MAQSLRLHFA ARRSNTYPLS ETSGDDLDSH VHMCFKRPTR ISTSNVVQMK | 50 |
| LTPRQTALAP LIKENVQSQE RSSVPSSENV NKKSSCLQIS LQPTRYSGYL | 100 |
| QSSNVLADSD DASFTCILKD GIYSSAVVDN ELNAVNDGHL VSSPAICSGS | 150 |
| LSNFSTSDNG SYSSNGSDFG SCASITSGGS YTNSVISDSS SYTFPPSDDT | 200 |
| FLGGNLPSDS TSNRSVPNRN TTPCEIFSRS TSTDPFVQDD LEHGLEIMKL | 250 |
| PVSRNTKIPL KRYSSLVIFP RSPSTTRPTS PTSLCTLLSK GSYQTSHQFI | 300 |
| ISPSEIAHNE DGTSAKGFLS TAVNGLRLSK TICTPGEVRD IRPLHRKGSL | 350 |
| QKKIVLSNNT PRQTVCEKSS EGYSCVSVHF TQRKAATLDC ETTNGDCKPE | 400 |
| MSEIKLNSDS EYIKLMHRTS ACLPSSQNVD CQININGELE RPHSQMNKNH | 450 |
| GILRRSISLG GAYPNISCLS SLKHNCSKGG PSQLLIKFAS GNEGKVDNLS | 500 |
| RDSNRDCTNE LSNSCKTRDD FLGQVDVPLY PLPTENPRLE RPYTFKDFVL | 550 |
| HPRSHKSRVK GYLRLKMTYL PKTSGSEDDN AEQAEELEPG WVVLDQPDAA | 600 |
| CHLQQQQEPS PLPPGWEERQ DILGRTYYVN HESRRTQWKR PTPQDNLTDA | 650 |
| ENGNIQLQAQ RAFTTRRQIS EETESVDNRE SSENWEIIRE DEATMYSNQA | 700 |
| FPSPPPSSNL DVPTHLAEEL NARLTIFGNS AVSQPASSSN HSSRRGSLQA | 750 |
| YTFEEQPTLP VLLPTSSGLP PGWEEKQDER GRSYYVDHNS RITTWTKPTV | 800 |
| QATVETSQLT SSQSSAGPQS QASTSDSGQQ VTQPSEIEQG FLPKGWEVRH | 850 |
| APNGRPFFID HNTKTTTWED PRLKIPAHLR GKTSLDTSND LGPLPPGWEE | 900 |
| RTHIDGRIFY INHNIKRTQW EDPRLENVAI TGPAVPYSRD YKRKYEFFRR | 950 |
| KLKKQNDIPN KFEMKIRRAT VLEDSYRRIM GVKRADFLKA RLWIEFDGEK | 1000 |
| GLDYGGVARE WFFLISKEMF NPYYGLFEYS ATDNYTLQIN PNSGLCNEDH | 1050 |
| LSYFKFIGRV AGMAVYHGKL LDGFFIRPFY KMMLHKPITL HDMESVDSEY | 1100 |
| YNSLRWILEN DPTELDLRFI IDEELFGQTH QHELKNGGSE IVVTNKNKKE | 1150 |
| YIYLVIQWRF VNRIQKQMAA FKEGFFELIP QDLIKIFDEN ELELLMCGLG | 1200 |
| DVDVNDWREH TKYKNGYSAN HQVIQWFWKA VLMMDSEKRI RLLQFVTGTS | 1250 |
| RVPMNGFAEL YGSNGPQSFT VEQWGTPEKL PRAHTCFNRL DLPPYESFEE | 1300 |
| LWDKLQMAIE NTQGFDGVD | 1319 |

WW1 (610-643):
(SEQ ID NO: 36)
SPLPPGWEERQDILGRTYYVNHESRRTQWKRPTP.
WW2 (767-800):
(SEQ ID NO: 37)
SGLPPGWEEKQDERGRSYYVDHNSRTTTWTKPTV.
WW3 (840-873):
(SEQ ID NO: 38)
GFLPKGWEVRHAPNGRPFFIDHNTKTTTWEDPRL.
WW4 (892-925):
(SEQ ID NO: 39)
GPLPPGWEERTHTDGRIFYINHNIKRTQWEDPRL.

Human Nedd4-2 amino acid sequence (>giI21361472IrefINP_056092.21E3 ubiquitin-protein ligase NEDD4-like isoform 3 [Homo sapiens]). The four underlined WW domains correspond to amino acids 198-224 (WW1), 368-396 (WW2), 480 — 510 (WW3), and 531 — 561 (WW4).

(SEQ ID NO: 40)
MATGLGEPVYGLSEDEGESRILRVKVVSGIDLAKKDIFGASDPYVKLSLYVADENRELALVQ
TKTIKKTLNPKWNEEFYFRVNPSNHRLLFEVFDENRLTRDDFLGQVDVPLSHLPTEDPTMER
PYTFKDFLLRPRSHKSRVKGFLRLKMAYMPKNGGQDEENSDQRDDMEHGWEVVDSNDSASQH
QEELPPPPLPPGWEEKVDNLGRTYYVNHNNRTTQWHRPSLMDVSSESDNNIRQINQEAAHRR
FRSRRHISEDLEPEPSEGGDVPEPWETISEEVNIAGDSLGLALPPPPASPGSRTSPQELSEE
LSRRLQITPDSNGEQFSSLIQREPSSRLRSCSVTDAVAEQGHLPPPSVAYVHTTPGLPSGWE
ERKDAKGRTYYVNHNNRTTTWTRPIMQLAEDGASGSATNSNNHLIEPQIRRPRSLSSPTVTL
SAPLEGAKDSPVRRAVKDTLSNPQSPQPSPYNSPKPQHKVTQSFLPPGWEMRIAPNGRPFFI
DHNTKTTTWEDPRLKFPVHMRSKTSLNPNDLGPLPPGWEERIHLDGRTFYIDHNSKITQWED
PRLQNPAITGPAVPYSREFKQKYDYFRKKLKKPADIPNRFEMKLHRNNIFEESYRRIMSVKR
PDVLKARLWIEFESEKGLDYGGVAREWFFLLSKEMENPYYGLFEYSATDNYTLQINPNSGLC
NEDHLSYFTFIGRVAGLAVFHGKLLDGFFIRPFYKMMLGKQITLNDMESVDSEYYNSLKWIL
ENDPTELDLMFCIDEENFGQTYQVDLKPNGSEIMVTNENKREYIDLVIQWRFVNRVQKQMNA
FLEGFTELLPIDLIKIFDENELELLMCGLGDVDVNDWRQHSIYKNGYCPNHPVIQWFWKAVL
LMDAEKRIRLLQFVTGTSRVPMNGFAELYGSNGPQLFTIEQWGSPEKLPRAHTCFNRLDLPP
YETFEDLREKLLMAVENAQGFEGVD
WW1 (198-224):
(SEQ ID NO: 41)
GWEEKVDNLGRTYYVNHNNRTTQWHRP.
WW2 (368-396):
(SEQ ID NO: 42)
PSGWEERKDAKGRTYYVNHNNRTTTWTRP.
WW3 (480-510):
(SEQ ID NO: 43)
PPGWEMRIAPNGRPFFIDHNTKTTTWEDPRL.
WW4 (531-561):
(SEQ ID NO: 44)
PPGWEERIHLDGRTFYIDHNSKITQWEDPRL.

Human Smurf1 amino acid sequence (uniprot.org/uniprot/Q9HCE7). The two underlined WW domains correspond to amino acids 234-267 (WW1) and 306-339 (WW2).

| (SEQ ID NO: 45) | |
|---|---|
| MSNPGTRRNG SSIKIRLTVL CAKNLAKKDF FRLPDPFAKI VVDGSGQCHS | 50 |
| TDTVKNTLDP KWNQHYDLYV GKTDSITISV WNHKKIHKKQ GAGFLGCVRL | 100 |
| LSNAISRLKD TGYQRLDLCK LNPSDTDAVR GQIVVSLQTR DRIGTGGSVV | 150 |
| DCRGLLENEG TVYEDSGPGR PLSCFMEEPA PYTDSTGAAA GGGNCRFVES | 200 |
| PSQDQRLQAQ RLRNPDVRGS LQTPQNRPHG HQSPELPEGY EQRTTVQGQV | 250 |
| YFLHTQTGVS TWHDPRIPSP SGTIPGGDAA FLYEFLLQGH TSEPRDLNSV | 300 |
| NCDELGPLPP GWEVRSTVSG RIYFVDHNNR TTQFTDPRLH HIMNHQCQLK | 350 |
| EPSQPLPLPS EGSLEDEELP AQRYERDLVQ KLKVLRHELS LQQPQAGHCR | 400 |
| IEVSREEIFE ESYRQIMKMR PKDLKKRLMV KERGEEGLDY GGVAREWLYL | 450 |
| LCHEMLNPYY GLFQYSTDNI YMLQINPDSS INPDHLSYFH FVGRIMGLAV | 500 |
| FHGHYINGGF TVPFYKQLLG KPIQLSDLES VDPELHKSLV WILENDITPV | 550 |
| LDHTFCVEHN AFGRILQHEL KPNGRNVPVT EENKKEYVRL YVNWRFMRGI | 600 |
| EAQFLALQKG FNELIPQHLL KPFDQKELEL IIGGLDKIDL NDWKSNTRLK | 650 |
| HCVADSNIVR WFWQAVETED EERRARLLQF VTGSTRVPLQ GFKALQGSTG | 700 |
| AAGPRLFTIH LIDANTDNLP KAHTCFNRID IPPYESYEKL YEKLLTAVEE | 750 |
| TCGFAVE | 757 |

WW1 (234-267):
(SEQ ID NO: 46)
PELPEGYEQRTTVQGQVYFLHTQTGVSTWHDPRI.
WW2 (306-339):
(SEQ ID NO: 47)
GPLPPGWEVRSTVSGRIYFVDHNNRTTQFTDPRL.

Human Smurf2 amino acid sequence (uniprot.org/uniprot/Q9HAU4). The three underlined WW domains correspond to amino acids 157-190 (WW1), 251-284 (WW2), and 297-330 (WW3).

| (SEQ ID NO: 48) | |
|---|---|
| MSNPGGRRNG PVKLRLTVLC AKNLVKKDFF RLPDPFAKVV VDGSGQCHST | 50 |
| DTVKNTLDPK WNQHYDLYIG KSDSVTISVW NHKKIHKKQG AGFLGCVRLL | 100 |
| SNAINRLKDT GYQRLDLCKL GPNDNDTVRG QIVVSLQSRD RIGTGGQVVD | 150 |
| CSRLFDNDLP DGWEERRTAS GRIQYLNHIT RTTQWERPTR PASEYSSPGR | 200 |
| PLSCFVDENT PISGTNGATC GQSSDPRLAE RRVRSQRHRN YMSRTHLHTP | 250 |
| PDLPEGYEQR TTQQGQVYFL HTQTGVSTWH DPRVPRDLSN INCEELGPLP | 300 |
| PGWEIRNTAT GRVYFVDHNN RTTQFTDPRL SANLHLVLNR QNQLKDQQQQ | 350 |
| QVVSLCPDDT ECLTVPRYKR DLVQKLKILR QELSQQQPQA GHCRIEVSRE | 400 |
| EIFEESYRQV MKMRPKDLWK RLMIKFRGEE GLDYGGVARE WLYLLSHEML | 450 |
| NPYYGLFQYS RDDIYTLQIN PDSAVNPEHL SYFHFVGRIM GMAVFHGHYI | 500 |
| DGGFTLPFYK QLLGKSITLD DMELVDPDLH NSLVWILEND ITGVLDHTFC | 550 |
| VEHNAYGEII QHELKPNGKS IPVNEENKKE YVRLYVNWRF LRGIEAQFLA | 600 |
| LQKGENEVIP QHLLKTEDEK ELELIICGLG KIDVNDWKVN TRLKHCTPDS | 650 |
| NIVKWFWKAV EFFDEERRAR LLQFVTGSSR VPLQGFKALQ GAAGPRLFTI | 700 |
| HQIDACTNNL PKAHTCFNRI DIPPYESYEK LYEKLLTAIE ETCGFAVE | 748 |

WW1 (157-190):
(SEQ ID NO: 49)
NDLPDGWEERRTASGRIQYLNHITRTTQWERPTR.
WW2 (251-284):
(SEQ ID NO: 50)
PDLPEGYEQRTTQQGQVYFLHTQTGVSTWHDPRV.
WW3 (297-330):
(SEQ ID NO: 51)
GPLPPGWEIRNTATGRVYFVDHNNRTTQFTDPRL.

Human ITCH amino acid sequence (uniprot.org/uniprot/Q96J02). The four underlined WW domains correspond to amino acids 326-359 (WW1), 358-391 (WW2), 438-471 (WW3), and 478-511 (WW4).

| (SEQ ID NO: 1) | |
|---|---|
| MSDSGSQLGS MGSLTMKSQL QITVISAKLK ENKKNWFGPS PYVEVTVDGQ | 50 |
| SKKTEKCNNT NSPKWKQPLT VIVTPVSKLH FRVWSHQTLK SDVLLGTAAL | 100 |
| DIYETLKSNN MKLEEVVVTL QLGGDKEPTE TIGDLSICLD GLQLESEVVT | 150 |
| NGETTCSENG VSLCLPRLEC NSAISAHCNL CLPGLSDSPI SASRVAGFTG | 200 |
| ASQNDDGSRS KDETRVSTNG SDDPEDAGAG ENRRVSGNNS PSLSNGGFKP | 250 |
| SRPPRPSRPP PPTPRRPASV NGSPSATSES DGSSTGSLPP TNTNTNTSEG | 300 |
| ATSGLIIPLT ISGGSGPRPL NPVTQAPLPP GWEQRVDQHG RVYYVDHVEK | 350 |
| RTTWDRPEPL PPGWERRVDN MGRIYYVDHF TRITTWQRPT LESVRNYEQW | 400 |
| QLQRSQLQGA MQQFNQRFIY GNQDLFATSQ SKEFDPLGPL PPGWEKRTDS | 450 |
| NGRVYFVNHN TRITQWEDPR SQGQLNEKPL PEGWEMRFTV DGIPYFVDHN | 500 |
| RRTTTYIDPR TGKSALDNGP QIAYVRDFKA KVQYFRFWCQ QLAMPQHIKI | 550 |
| TVTRKTLFED SFQQIMSFSP QDLRRRLWVI FPGEEGLDYG GVAREWFFLL | 600 |
| SHEVLNPMYC LFEYAGKDNY CLQINPASYI NPDHLKYFRF IGRFIAMALF | 650 |
| HGKFIDTGFS LPFYKRILNK PVGLKDLESI DPEFYNSLIW VKENNIEECD | 700 |
| LEMYFSVDKE ILGEIKSHDL KPNGGNILVT EENKEEYIRM VAEWRLSRGV | 750 |
| EEQTQAFFEG FNEILPQQYL QYFDAKELEV LLCGMQEIDL NDWQRHAIYR | 800 |
| HYARTSKQIM WFWQFVKEID NEKRMRLLQF VTGTCRLPVG GFADLMGSNG | 850 |
| PQKFCIEKVG KENWLPRSHT CFNRLDLPPY KSYEQLKEKL LFAIEETEGF | 900 |
| GQE | 903 |

ITCH WW1 (326-359):
(SEQ ID NO: 13)
APLPPGWEQRVDQHGRVYYVDHVEKRTTWDRPEP.
ITCH WW2 (358-391):
(SEQ ID NO: 14)
EPLPPGWERRVDNMGRIYYVDHFTRTTTWQRPTL.
ITCH WW3 (438-471):
(SEQ ID NO: 4)
GPLPPGWEKRTDSNGRVYFVNHNTRITQWEDPRS.
ITCH WW4 (478-511):
(SEQ ID NO: 6)
KPLPEGWEMRFTVDGIPYFVDHNRRTTTYIDPRT.

Human NEDL1 amino acid sequence (uniprot.org/uniprot/Q76N89). The two underlined WW domains correspond to amino acids 829-862 (WW1), and 1018-1051 (WW2).

| (SEQ ID NO: 52) | |
|---|---|
| MLLHLCSVKN LYQNRFLGLA AMASPSRNSQ SRRRCKEPLR YSYNPDQFHN | 50 |
| MDLRGGPHDG VTIPRSISDT DLVISDSRST LMVSSSYYSI GHSQDLVIHW | 100 |
| DIKEEVDAGD WIGMYLIDEV LSENFLDYKN RGVNGSHRGQ IIWKIDASSY | 150 |
| FVEPETKICF KYYHGVSGAL RATTPSVTVK NSAAPIFKSI GADETVQGQG | 200 |
| SRRLISFSLS DFQAMGLKKG MFFNPDPYLK ISIQPGKHSI FPALPHHGQE | 250 |
| RRSKIIGNTV NPIWQAEQFS FVSLPTDVLE IEVKDKFAKS RPIIKRFLGK | 300 |
| LSMPVQRLLE RHAIGDRVVS YTLGRRLPTD HVSGQLQFRF EITSSIHPDD | 350 |
| EEISLSTEPE SAQIQDSPMN NLMESGSGEP RSEAPESSES WKPEQLGEGS | 400 |
| VPDGPGNQSI ELSRPAEEAA VITEAGDQGM VSV GPEGAGE LLAQVQKDIQ | 450 |
| PAPSAEELAE QLDLGEEASA LLLEDGEAPA STKEEPLEEE ATTQSRAGRE | 500 |
| EEEKEQEEEG DVSTLEQGEG RLQLRASVKR KSRPCSLPVS ELETVIASAC | 550 |
| GDPETPRTHY IRIHTLLHSM PSAQGGSAAE EEDGAEEEST LKDSSEKDGL | 600 |
| SEVDTVAADP SALEEDREEP EGATPGTAHP GHSGGHFPSL ANGAAQDGDT | 650 |
| HPSTGSESDS SPRQGGDHSC EGCDASCCSP SCYSSSCYST SCYSSSCYSA | 700 |
| SCYSPSCYNG NRFASHTRES SVDSAKISES TVFSSQDDEE EENSAFESVP | 750 |
| DSMQSPELDP ESTNGAGPWQ DELAAPSGHV ERSPEGLESP VAGPSNRREG | 800 |
| ECPILHNSQP VSQLPSLRPE HHHYPTIDEP LPPNWEARID SHGRVFYVDH | 850 |
| VNRITTWQRP TAAATPDGMR RSGSIQQMEQ LNRRYQNIQR TIATERSEED | 900 |
| SGSQSCEQAP AGGGGGGGSD SEAESSQSSL DLRREGSLSP VNSQKITLLL | 950 |
| QSPAVKFITN PEFFTVLHAN YSAYRVFTSS TCLKHMILKV RRDARNFERY | 1000 |
| QHNRDLVNFI NMFADTRLEL PRGWEIKTDQ QGKSFFVDHN SRATTFIDPR | 1050 |
| IPLQNGRLPN HLTHRQHLQR LRSYSAGEAS EVSRNRGASL LARPGHSLVA | 1100 |
| AIRSQHQHES LPLAYNDKIV AFLRQPNIFE MLQERQPSLA RNHTLREKIH | 1150 |
| YIRTEGNHGL EKLSCDADLV ILLSLFEEEI MSYVPLQAAF HPGYSFSPRC | 1200 |
| SPCSSPQNSP GLQRASARAP SPYRRDFEAK LRNFYRKLEA KGFGQGPGKI | 1250 |
| KLIIRRDHLL EGTFNQVMAY SRKELQRNKL YVTFVGEEGL DYSGPSREFF | 1300 |
| FLLSQELFNP YYGLFEYSAN DTYTVQISPM SAFVENHLEW FRFSGRILGL | 1350 |
| ALIHQYLLDA FFTRPFYKAL LRLPCDLSDL EYLDEEFHQS LQWMKDNNIT | 1400 |
| DILDLTFTVN EEVFGQVTER ELKSGGANTQ VTEKNKKEYI ERMVKWRVER | 1450 |
| GVVQQTEALV RGFYEVVDSR LVSVFDAREL ELVIAGTAEI DLNDWRNNTE | 1500 |
| YRGGYHDGHL VIRWFWAAVE RFNNEQRLRL LQFVTGTSSV PYEGFAALRG | 1550 |
| SNGLRRFCIE KWGKITSLPR AHTCFNRLDL PPYPSYSMLY EKLLTAVEET | 1600 |
| STFGLE | 1606 |

WW1 (829-862):
(SEQ ID NO: 53)
PLPPNWEARIDSHGRVFYVDHVNRTTTWQRPTA.
WW2(1018-1051):
(SEQ ID NO: 54)
LELPRGWEIKTDQQGKSFFVDHNSRATTFIDPRI.

Human NEDL2 amino acid sequence (uniprot.org/uniprot/Q9P2P5). The two underlined WW domains correspond to amino acids 807-840 (WW1) and 985-1018 (WW2).

| (SEQ ID NO: 55) | |
|---|---|
| MASSAREHLL FVRRRNPQMR YILSPENLQS LAAQSSMPEN MTLQRANSDT | 50 |
| DLVTSESRSS LTASMYEYTL GQAQNLIIFW DIKEEVDPSD WIGLYHIDEN | 100 |
| SPANFWDSKN RGVIGTQKGQ IVWRIEPGPY FMEPEIKICF KYYHGISGAL | 150 |
| RATTPCITVK NPAVMMGAEG MEGGASGNLH SRKLVSFTLS DLRAVGLKKG | 200 |
| MFFNPDPYLK MSIQPGKKSS FPTCAHHGQE RRSTIISNIT NPIWHREKYS | 250 |
| FFALLTDVLE IEIKDKFAKS RPIIKRFLGK LTIPVQRLLE RQAIGDQMLS | 300 |
| YNLGRRLPAD HVSGYLQFKV EVISSVHEDA SPEAVGTILG VNSVNGDLGS | 350 |
| PSDDEDMPGS HHDSQVCSNG PVSEDSAADG TPKHSFRTSS TLEIDTEELT | 400 |
| STSSRTSPPR GRQDSLNDYL DAIEHNGHSR PGTATCSERS MGASPKLRSS | 450 |
| FPTDTRLNAM LHIDSDEEDH EFQQDLGYPS SLEEEGGLIM FSRASRADDG | 500 |
| SLTSQTKLED NPVENEEAST HEAASFEDKP ENLPELAESS LPAGPAPEEG | 550 |
| EGGPEPQPSA DQGSAELCGS QEVDQPTSGA DTGTSDASGG SRRAVSETES | 600 |
| LDQGSEPSQV SSETEPSDPA RTESVSEAST RPEGESDLEC ADSSCNESVT | 650 |
| TQLSSVDTRC SSLESARFPE TPAFSSQEEE DGACAAEPTS SGPAEGSQES | 700 |
| VCTAGSLPVV QVPSGEDEGP GAESATVPDQ EELGEVWQRR GSLEGAAAAA | 750 |
| ESPPQEEGSA GEAQGTCEGA TAQEEGATGG SQANGHQPLR SLPSVRQDVS | 800 |
| RYQRVDEALP PNWEARIDSH GRIFYVDHVN RTTTWQRPTA PPAPQVLORS | 850 |
| NSIQQMEQLN RRYQSIRRTM TNERPEENTN AIDGAGEEAD FHQASADFRR | 900 |
| ENILPHSTSR SRITLLLQSP PVKFLISPEF FTVLHSNPSA YRMFTNNTCL | 950 |
| KHMITKVRRD THHFERYQHN RDLVGFLNMF ANKQLELPRG WEMKHDHQGK | 1000 |
| AFFVDHNSRT TTFIDPRLPL QSSRPTSALV HRQHLTRQRS HSAGEVGEDS | 1050 |
| RHAGPPVLPR PSSTFNTVSR PQYQDMVPVA YNDKIVAFLR QPNIFEILQE | 1100 |
| RQPDLTRNHS LREKIQFIRT EGTPGLVRLS SDADLVMLLS LFEEEIMSYV | 1150 |
| PPHALLHPSY CQSPRGSPVS SPQNSPGTQR ANARAPAPYK RDFEAKLRNF | 1200 |
| YRKLETKGYG QGPGKLKLII RRDHLLEDAF NQIMGYSRKD LQRNKLYVTE | 1250 |
| VGEEGLDYSG PSREFFFLVS RELFNPYYGL FEYSANDTYT VQISPMSAFV | 1300 |
| DNHHEWFRFS GRILGLALIH QYLLDAFFTR PFYKALLRIL CDLSDLEYLD | 1350 |
| EEFHQSLQWM KDNDIHDILD LTFTVNEEVF GQITERELKP GGANIPVTEK | 1400 |
| NKKEYIERMV KWRIERGVVQ QTESLVRGFY EVVDARLVSV FDARELELVI | 1450 |
| AGTAEIDLSD WRNNTEYRGG YHDNHIVIRW FWAAVERFNN EQRLRLLQFV | 1500 |
| TGTSSIPYEG FASLRGSNGP RRFCVEKWGK ITALPRAHTC FNRLDLPPYP | 1550 |
| SFSMLYEKLL TAVEETSTEG LE | 1572 |

WW1 (807-840):
(SEQ ID NO: 56)
EALPPNWEARIDSHGRIFYVDHVNRITTWQRPTA.
WW2 (985-1018):
(SEQ ID NO: 57)
LELPRGWEMKHDHQGKAFFVDHNSRTTTFIDPRL.

In some embodiments, the WW-containing domain consists essentially of a WW-containing domain or WW-containing domain variant. Consists essentially of means that a domain, peptide, or polypeptide consists essentially of an amino acid sequence when such an amino acid sequence is present with only a few additional amino acid residues, for example, from about 1 to about 10 or so additional residues, typically from 1 to about 5 additional residues in the domain, peptide, or polypeptide.

Alternatively, the WW-containing domain may be a WW-containing domain that has been modified to include two basic amino acids at the C-terminus of the domain. Techniques are known in the art and are described in the art, for example, in Sambrook et al., ((2001) Molecular Cloning: a Laboratory Manual, 3rd ed., Cold Spring Harbour Laboratory Press). Thus, a skilled person could readily modify an existing WW-containing domain that does not normally have two C-terminal basic residues so as to include two basic residues at the C terminus.

Basic amino acids are amino acids that possess a side-chain functional group that has a pKa of greater than 7 and includes lysine, arginine, and histidine, as well as basic amino acids that are not included in the twenty a-amino acids commonly included in proteins. The two basic amino acids at the C terminus of the WW-containing domain may be the same basic amino acid or may be different basic amino acids. In one embodiment, the two basic amino acids are two arginine residues.

The term WW-containing domain also includes variants of a WW-containing domain provided that any such variant possesses two basic amino acids at its C-terminus and maintains the ability of the WW-containing domain to associate with the PPXY (SEQ ID NO: 22) motif. A variant of such a WW-containing domain refers to a WW-containing domain which retains the ability of the variant to associate with the PPXY (SEQ ID NO: 22) motif (i.e., the PPXY (SEQ ID NO: 22) motif of SCAMP3 and that has been mutated at one or more amino acids, including point, insertion, and/or deletion mutations, but still retains the ability to associate with the PPXY (SEQ ID NO: 22) motif. A variant or derivative therefore includes deletions, including truncations and fragments; insertions and additions, for example conservative substitutions, site-directed mutants and allelic variants; and modifications, including one or more non-amino acyl groups (e.g., sugar, lipid, etc.) covalently linked to the peptide and post-translational modifications. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

The WW-containing domain may be part of a longer protein. Thus, the protein, in various different embodiments, comprises the WW-containing domain, consists of the WW-containing domain or consists essentially of the WW-containing domain, as defined herein. The polypeptide may be a protein that includes a WW domain as a functional domain within the protein sequence.

### DETAILED DESCRIPTION OF CERTAIN EMBODIMENTS OF THE INVENTION

The present disclosure relates, at least in part, to novel extracellular vesicles (EVs) which contain WW-domain containing proteins that comprise an extracellular domain (WW-domain-Activated Extracellular Vesicles, or WAEVs). Such extracellular domains can be presented on the surface of the WAEV through the introduction of WW-domain containing proteins that are fused to a transmembrane domain and the extracellular domain. Direct fusions of transmembrane-containing proteins to arrestin domain containing protein 1 (ARDDC1) result in decreased or abolished budding activity of ARRCC1. WAEVs are able to bud independent of ARRDC1, and do not appear to be enhanced by ARDDC1 overexpression. In addition, WAEVs do not appear to be like classical exosomes because they do not contain one or more of the typical exosomal markers (e.g., CD63; CD81, CD9, and PTGFRN). Instead, other proteins may be responsible for mediating WAEV budding, including the secretory carrier-associated membrane protein 3 (SCAMP3). WAEVs can be used to deliver and present viral or bacterial antigens useful for vaccine development; to display homing molecules for targeted delivery of therapeutic molecules to specific cells or tissues; and for packaging and delivery of therapeutic molecules via interactions with the WW domains.

### WW-Domain-Activated Extracellular Vesicles (WAEVs)

In some aspects, the disclosure relates to a WW-domain-activated extracellular vesicle (WAEV), comprising: (a) a lipid bilayer; and (b) a fusion protein as described herein.

In some embodiments, a WAEV as described herein, further comprises WAEV-mediating protein. WAEV-mediating proteins can contain either the PPXY (SEQ ID NO: 22) motif or the PSAP (SEQ ID NO: 17) motif, and preferably contain both. The PPXY (SEQ ID NO: 22) and PSAP (SEQ ID NO: 17) motifs are critical elements in the ARDDC1 protein that are required for ARMMs budding. The WAEV-mediating protein can interact with fusion proteins WW-containing domain through the PPXY (SEQ ID NO: 22) motif, and the WAEV-mediating protein can recruit TSG101 via the PSAP (SEQ ID NO: 17) motif to the cell membrane to drive the budding of WAEVs.

A non-limiting example of a WAEV-mediating protein is SCAMP3. Secretory carrier-associated membrane protein 3 (SCAMP3) is a protein that in humans is encoded by the SCAMP3 gene, which is a member of the SCAMP family of proteins that are secretory carrier membrane proteins. These proteins are known to function as carriers of proteins to the cell surface in post-golgi recycling pathways. SCAMP3 is an integral membrane protein that has four transmembrane domains and contains a PPXY (SEQ ID NO: 22) motif at its N-terminal cytosolic segment. In addition, SCAMP3 has a PSAP (SEQ ID NO: 17) motif that is known to interact with TSG101, the ESCRT I complex protein required for budding of ARMMs (see U.S. Pat. Ser. No. 9,737,480) as well as other multivesicular bodies. Thus, SCAMP3 shares both PPXY (SEQ ID NO: 22) and PSAP (SEQ ID NO: 17) motif with ARRDC1 but differs from ARRDC1 in that SCAMP3 is integrated in the plasma membrane via its transmembrane domain whereas ARRDC1 transiently associates with plasma membrane via its arrestin domain. It is believed that the that fusion protein WW-containing domain (e.g., WW-containing domain protein fused to a transmembrane domain and extracellular domain) interacts with the PPXY (SEQ ID NO: 22) motif of SCAMP3, which subsequently recruits TSG101 via the PSAP (SEQ ID NO: 17) motif to the cell membrane to drive the budding of WAEVs. The extracellular domain can include a cargo domain.

Tumor susceptibility gene 101 (TSG101), refers to a group of seemingly inactive homologs of ubiquitin-conjugating enzymes. The protein contains a coiled-coil domain that interacts with stathmin, a cytosolic phosphoprotein implicated in tumorigenesis. TSG101 can interact with proteins that comprises a PSAP (SEQ ID NO: 17) motif. TSG101, in budding viruses, drives budding through direct plasma membrane budding (DPMB). TSG101 is a protein that comprises a UEV domain and can interact with SCAMP3. As referred to herein, UEV refers to the Ubiquitin E2 variant domain of approximately 145 amino acids. The structure of the domain contains a a/f3 fold similar to the canonical E2 enzyme but has an additional N-terminal helix and further lacks the two C-terminal helices. Often found in TSG101/Vps23 proteins, the UEV interacts with a ubiquitin molecule and is essential for the trafficking of a number of ubiquitylated payloads to multivesicular bodies (MVBs). Furthermore, the UEV domain can bind to Pro-Thr/Ser-Ala-Pro peptide ligands, a fact exploited by viruses such as HIV. Thus, the TSG101 UEV domain binds to the PTAP tetrapeptide motif in the viral Gag protein that is involved in viral budding. The disclosure also contemplates variants of TSG101, such as fragments of TSG101 and/or TSG101 proteins that have a degree of identity (e.g., 60%, 70%, 80%, 85%, 90%, 95%, 98%, or 99% identity) to a TSG101 protein and are capable of interacting with PSAP-containing proteins like SCAMP3. Accordingly, an TSG101 protein may be a protein that comprises a UEV domain and interacts with SCAMP3. In some embodiments, the TSG101 protein is at least 60%, 65%, 70%, 75%, 80%, 85%, 90%, 95%, 98%, or 99% identical to the amino acid sequence of any one of SEQ ID NO: 58, comprises a UEV domain, and interacts with PSAP-containing proteins like SCAMP3. In some embodiments, the TSG101 protein has at least 10, at least at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, or at least 390, identical contiguous amino acids of any one of SEQ ID NO: 58, comprises a UEV domain, and interacts PSAP-containing proteins like SCAMP3. In some embodiments, the TSG101 protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NO: 58 and comprises a UEV domain. Exemplary, non-limiting TSG101 protein sequences are provided herein, and additional, suitable TSG101 protein sequences, isoforms, and variants are known in the art. It will be appreciated by those of skill in the art that this invention is not limited in this respect. Exemplary TSG101 sequences include the following sequences (the UEV domain in these sequences includes amino acids 1-145 and is underlined in the sequences below):

>gi|5454140|ref|NP_006283.1| tumor susceptibility gene 101
protein [Homo sapiens]
(SEQ ID NO: 58)
MAVSESQLKKMVSKYKYRDLTVRETVNVITLYKDLKPVLDSYVFNDGSSRE
LMNLTGTIPVPYRGNTYNIPICLWLLDTYPYNPPICFVKPTSSMTIKTGKHVDANGKI
YLPYLHEWKHPQSDLLGLIQVMIVVFGDEPPVFSRPISASYPPYQATGPPNTSYMPGM
PGGISPYPSGYPPNPSGYPGCPYPPGGPYPATTSSQYPSQPPVTTVGPSRDGTISEDTIR
ASLISAVSDKLRWRMKEEMDRAQAELNALKRTEEDLKKGHQKLEEMVTRLDQEVA
EVDKNIELLKKKDEELSSALEKMENQSENNDIDEVIIPTAPLYKQILNLYAEENAIEDT
IFYLGEALRRGVIDLDVFLKHVRLLSRKQFQLRALMQKARKTAGLSDLY
>gi|11230780|ref|NP_068684.1| tumor susceptibility gene 101
protein [Mus musculus]
(SEQ ID NO: 59)
MAVSESQLKKMMSKYKYRDLTVRQTVNVIAMYKDLKPVLDSYVENDGSSR
ELVNLTGTIPVRYRGNIYNIPICLWLLDTYPYNPPICFVKPTSSMTIKTGKHVDANGKI
YLPYLHDWKHPRSELLELIQIMIVIFGEEPPVFSRPTVSASYPPYTATGPPNTSYMPGM
PSGISAYPSGYPPNPSGYPGCPYPPAGPYPATTSSQYPSQPPVTTVGPSRDGTISEDTIR
ASLISAVSDKLRWRMKEEMDGAQAELNALKRTEEDLKKGHQKLEEMVTRLDQEVA
EVDKNIELLKKKDEELSSALEKMENQSENNDIDEVIIPTAPLYKQILNLYAEENAIEDT
IFYLGEALRRGVIDLDVFLKHVRLLSRKQFQLRALMQKARKTAGLSDLY
>gi|48374087|ref|NP_853659.2| tumor susceptibility gene 101
protein [Rattus norvegicus]
(SEQ ID NO: 60)
MAVSESQLKKMMSKYKYRDLTVRQTVNVIAMYKDLKPVLDSYVFNDGSSR
ELVNLTGTIPVRYRGNIYNIPICLWLLDTYPYNPPICFVKPTSSMTIKTGKHVDANGKI
YLPYLHDWKHPRSELLELIQIMIVIFGEEPPVFSRPTVSASYPPYTAAGPPNTSYLPSM
PSGISAYPSGYPPNPSGYPGCPYPPAGPYPATTSSQYPSQPPVTTAGPSRDGTISEDTIR
ASLISAVSDKLRWRMKEEMDGAQAELNALKRTEEDLKKGHQKLEEMVTRLDQEVA
EVDKNIELLKKKDEELSSALEKMENQSENNDIDEVIIPTAPLYKQILNLYAEENAIEDT
IFYLGEALRRGVIDLDVFLKHVRLLSRKQFQLRALMQKARKTAGLSDLY.

The structure of UEV domains is known to those of skill in the art (see, e.g., Owen Pornillos et al., Structure and functional interactions of the Tsg101 UEV domain, EMBO J. 2002 May 15; 21(10): 2397-2406, the entire contents of which are incorporated herein by reference).

In some embodiments, the fusion proteins of the disclosure do not comprise an arrestin domain containing protein 1 (ARRDC1). ARRDC1, as described elsewhere herein, is a protein that comprises a PSAP (SEQ ID NO: 17) motif and a PPXY (SEQ ID NO: 22) motif in its C-terminus and interacts with TSG101. However, as can be shown herein, the present WAEVs do not require the presence or action of ARRDC1 to form and/or bud. Accordingly, in some embodiments, the WAEVs of the present disclosure lack an ARRDC1 protein.

The WAEVs of the present disclosure further are distinguishable from various other exosomes and/or extracellular vesicles in markers they carry. Typical EVs carry a variety of proteins used as markers to identify exosomes, as well as imbue qualities to the exosome for use in experiments and diagnostics. Exosomal markers are known in the art, and are known, for example, to belong to various functional groups, such as tetraspanins (CD9, CD63 and CD81), heat shock proteins (HSC70 and HSC90), membrane transporters (GTPases) and lipid-bound proteins. Some of the most prevalent exosomal markers include: heat shock protein 8 (HSPA8), CD63 antigen (CD63), beta actin (ACTB), glyceraldehyde-3-phosphate dehydrogenase (GAPDH), enolase 1 alpha (ENO1), cytosolic heat shock protein 90 alpha (HSP90AA1), CD9, CD81, tyrosine 3-monooxygenase/tryptophan 5-monooxygenase activation protein, zeta polypeptide (YWHAZ), muscle pyruvate kinase (PKM2). However, the WAEVs of the present disclosure can lack one or more (or all) of the anticipated markers found in EVs, for example: CD9; CD63; CD81; and/or PTGFRN.

Accordingly, in some embodiments, the WAEVs of the present disclosure are enriched for a number of proteins.

TABLE 1

| | |
|---|---|
| ITCH | E3 ubiquitin-protein ligase itchy homolog OS = |
| | Homo sapiens GN = ITCH PE = 1 SV = 2 |
| PRKDC | DNA-dependent protein kinase catalytic subunit OS = |
| | Homo sapiens GN = PRKDC PE = 1 SV = 3 |
| DHX9 | ATP-dependent RNA helicase A OS = Homo sapiens GN = |
| | DHX9 PE = 1 SV = 4 |
| RUVBL1 | RuvB-like 1 OS = Homo sapiens GN = RUVBL1 PE = |
| | 1 SV = 1 |
| RUVBL2 | RuvB-like 2 OS = Homo sapiens GN = RUVBL2 PE = |
| | 1 SV = 3 |
| ESD | S-formylglutathione hydrolase OS = Homo sapiens GN = |
| | ESD PE = 1 SV = 2 |
| AMOT | Angiomotin OS = Homo sapiens GN = AMOT PE = 1 |
| | SV = 1 |
| ABCE1 | ATP-binding cassette sub-family E member 1 OS = |
| | Homo sapiens GN = ABCE1 PE = 1 SV = 1 |
| DARS | Aspartate-tRNA ligase, cytoplasmic OS = Homo sapiens |
| | GN = DARS PE = 1 SV = 2 |
| APOB | Apolipoprotein B-100 OS = Homo sapiens GN = |
| | APOB PE = 1 SV = 2 |
| RARS | Arginine-tRNA ligase, cytoplasmic OS = Homo sapiens |
| | GN = RARS PE = 1 SV = 2 |
| CLTC | Clathrin heavy chain 1 OS = Homo sapiens GN = |
| | CLTC PE = 1 SV = 5 |
| SCAMP3 | Secretory carrier-associated membrane protein 3 OS = |
| | Homo sapiens GN = SCAMP3 PE = 1 SV = 3 |
| FASN | Fatty acid synthase OS = Homo sapiens GN = |
| | FASN PE = 1 SV = 3 |
| ASNS | Asparagine synthetase [glutamine-hydrolyzing] OS = |
| | Homo sapiens GN = ASNS PE = 1 SV = 4 |
| DYNC1H1 | Cytoplasmic dynein 1 heavy chain 1 OS = Homo sapiens |
| | GN = DYNC1H1 PE = 1 SV = 5 |
| PSMD2 | 26S proteasome non-ATPase regulatory subunit 2 OS = |
| | Homo sapiens GN = PSMD2 PE = 1 SV = 3 |
| HSP90AB3P | Putative heat shock protein HSP 90-beta 3 OS = |
| | Homo sapiens GN = HSP90AB3P PE = 1 SV = 1 |
| HSP90AB2P | Putative heat shock protein HSP 90-beta 2 OS = |
| | Homo sapiens GN = HSP90AB2P PE = 1 SV = 2 |
| EPRS | Bifunctional glutamate/proline-tRNA ligase OS = |
| | Homo sapiens GN = EPRS PE = 1 SV = 5 |

In some embodiments, the WAEV comprises at least one enriched protein selected from Table 1. In some embodiments, the WAEV comprises at least two enriched proteins selected from Table 1. In some embodiments, the WAEV comprises at least three enriched proteins selected from Table 1. In some embodiments, the WAEV comprises at least four enriched proteins selected from Table 1. In some embodiments, the WAEV comprises at least five enriched proteins selected from Table 1. In some embodiments, the WAEV comprises more than five (e.g., enriched proteins selected from Table 1. In some embodiments, the WAEV comprises one or more proteins derived from one or more of the enriched protein selected from Table 1, including the WW-containing domains fusion proteins.

In some embodiments, the WAEVs as described herein do not comprise at least one of the following exosomal markers: CD9; CD63; CD81; and/or PTGFRN. In some embodiments, a WAEV as described herein does not comprise at least two of the following exosomal markers: CD9; CD63; CD81; and/or PTGFRN. In some embodiments, a WAEV as described herein does not comprise at least three of the following exosomal markers: CD9; CD63; CD81; and/or PTGFRN. In some embodiments, a WAEV as described herein does not comprise any of the following exosomal markers: CD9; CD63; CD81; and/or PTGFRN.

### Fusion Proteins

In some aspects, the disclosure relates to a fusion protein comprising: (a) a WW-containing domain; (b) a transmembrane domain; and (c) an extracellular domain. In some embodiments, the WW-containing domain is positioned at the N-terminus of the fusion protein. The fusion proteins of the present disclosure, may facilitate (e.g., increase the likelihood of, influence the production of) the production of WAEVs. In some embodiments, the fusion proteins, by containing extracellular domains as described in further detail herein, may facilitate the expression of, or presentation, of domains on the surface, or protruding from the surface of WAEVs.

Accordingly, in some embodiments, the WW-containing domain of any of the fusion proteins of the disclosure comprise at least one WW domain. In some embodiments, the WW-containing domain is positioned at the C-terminus of the fusion protein. In some embodiments, the WW-containing domain is positioned between the N-terminus and C-terminus of the fusion protein (e.g., between two other domains). In some embodiments, the WW-containing domain is positioned at the N-terminus of the fusion protein.

In some embodiments, the WW-containing domain of any of the fusion proteins of the disclosure comprise at least two WW domains. In some embodiments, the WW-containing domain of any of the fusion proteins of the disclosure comprise at least three WW domains. In some embodiments, the WW-containing domain of any of the fusion proteins of the disclosure comprise at least four WW domains. In some embodiments, the WW-containing domain of any of the fusion proteins of the disclosure comprise more than four WW domains. In some embodiments, the fusion protein comprises at least one WW domain which is an ITCH protein WW domain. In some embodiments, the WW-containing domain of any of the fusion proteins of the disclosure comprise a sequence having at least 95% identity to the sequence of SEQ ID NO: 1. In some embodiments, the WW-containing domain of any of the fusion proteins of the disclosure comprise the sequence of SEQ ID NO: 1. In some embodiments, where the WW-containing domain contains more than one WW domain, the WW domains may be oriented in the fusion protein such that they are adjacent to one another without another domain in between. In some embodiments, where the WW-containing domain contains more than one WW domain, the WW domains may be oriented in the fusion protein such that they are not adjacent to one another (e.g., with an intervening domain). In some embodiments, the intervening domain may be a linker domain. In some embodiments, the intervening domain may be another domain (e.g., peptide, molecule, nucleic acid). In some embodiments at least one of the WW domains of the fusion protein is positioned such that it has a free N-terminus. In some embodiments at least one of the WW domains of the fusion protein is positioned such that it has a free C-terminus.

In some embodiments, the fusion proteins of the disclosure comprise an extracellular domain. The extracellular domain is a portion (e.g., domain) of the fusion protein, which will be oriented (e.g., located, positioned), such that at least a portion of the extracellular domain is physically located outside of the membrane of the molecule (e.g., cell, EV) to which it is associated. In some embodiments, the entirety of the extracellular domain is located exterior to the membrane. In some embodiments, the extracellular domain comprises a known protein. In some embodiments, the extracellular domain comprises a portion of a known protein (e.g., fragment). In some embodiments, an extracellular domain of the fusion protein is the extracellular domain or a known protein, or fragment thereof. In some embodiments, the extracellular domain may be a recombinant protein, or fragment thereof (e.g., recombinant or engineered protein, fusion protein, or fragment thereof). In some embodiments, the extracellular domain may comprise a protein, or fragment thereof, which is known to provoke an immune response in an organism. In some embodiments, the extracellular domain may comprise a protein, or fragment thereof, which is believed to provoke an immune response in an organism. In some embodiments, the extracellular domain may comprise a protein, or fragment thereof, which is anticipated to provoke an immune response in an organism. In some embodiments, the extracellular domain may comprise a protein, or fragment thereof, which is desired to provoke an immune response in an organism. In some embodiments, the extracellular domain may comprise one or more carbohydrate unit that may or may not be responsible for, or involved in, provoking an immune response in an organism. In some embodiments, the extracellular domain may comprise one or more lipid unit that may or may not be responsible for, or involved in, provoking an immune response in an organism. As used herein, the term “provoke” is intended to describe a cause or impetus, the introduction of which into an organism influences or affects, at least in part, an immune reaction therein. Any action, beneficial or harmful (e.g., deleterious) is encompassed by the term. A direct reaction is not required (e.g., the reaction may be only partial caused by, or driven by, the introduction of the cause (e.g., domain, extracellular domain, protein, fusion protein), and may further be a component of, or step in, a larger cascade or reaction), nor must the reaction be substantial or complete. The immune reaction may further require the addition of other components.

In some embodiments, the extracellular domain comprises an antigen, or fragment thereof. In some embodiments, the extracellular domain of any of the fusion proteins of the disclosure comprises a viral antigen domain. In some embodiments, the viral antigen is a protein, or fragment thereof, from a respiratory virus. In some embodiments, the respiratory virus is selected from the group consisting of: adenovirus (ADV); influenza virus, human bocavirus (HBoV); human coronavirus (HCoV); human metapneumovirus (HMPV); human parainfluenza virus (HPIV); human respiratory syncytial virus (HRSV); human rhinovirus (HRV); severe acute respiratory syndrome coronavirus (SARS-CoV); and Middle East Respiratory Syndrome coronavirus (MERS-CoV). In some embodiments, the respiratory virus is a coronavirus. In some embodiments, the coronavirus is a SARS-CoV or a MERS-CoV. In some embodiments, the coronavirus is SARS-CoV-2. In some embodiments, the extracellular domain is a fragment of an antigen, or is derived from a virus or respiratory virus. In some embodiments, the coronavirus domain is a SARS-CoV-2-derived protein. In some embodiments, the coronavirus domain is the SARS-CoV-2 M protein, the SARS-CoV-2 E protein, or the SARS-CoV-2 S protein.

In some embodiments, the fusion proteins of the disclosure further comprise a signal or reporter. The reporter may be associated with the fusion protein in any way to facilitate the intended use of the reporter (e.g., detection or identification of the fusion protein). In some embodiments, the reporter is directly linked to the fusion protein. In some embodiments, the reporter is indirectly linked to the fusion protein. In some embodiments, the reporter is indirectly linked by a linker to the fusion protein. In some embodiments, the reporter is positioned at the N-terminus of the fusion protein. In some embodiments, the reporter is positioned at the C-terminus of the fusion protein. In some embodiments, the reporter is positioned internally of the fusion protein such that it is positioned between the domains of the fusion protein. In some embodiments, the reporter is GFP. In other embodiments, the reporter is mCherry, tdTomato, or any other fluorescence protein. In some embodiments, the report is luciferase or a recombinase, such as CRE or FLP.

### Nucleic Acids

In some embodiments, any of the isolated nucleic acids of the disclosure are operably linked to a promoter. In some embodiments, the promoter is a constitutive promoter, an inducible promoter, or a tissue specific promoter. In some embodiments, the promoter is a chicken beat-actin (CBA) promoter. In other embodiments, that promoter is EF-1-alpha. In some embodiments, the promoter is a viral promoter such as CMV, SV40. In some embodiments, the promoter is a prokaryotic promoter.

In some embodiments, any of the isolated nucleic acids of the disclosure comprise at least one additional regulatory sequence. In some embodiments, the regulatory sequence is an enhancer. In some embodiments, the regulatory sequence is a self-amplifying RNA.

### WAEV-Producing Cells

In some aspects, the disclosure relates to a WAEV-producing cell, comprising: (a) at least one of any of the isolated nucleic acids of the disclosure. In some embodiments, the WAEV-producing cell further comprises a heterologous promoter operably linked to a heterologous promoter. The WAEV-producing cell may be any type of suitable cell. For example, without limitation, the cell may be a target cell as described elsewhere herein. In some embodiments, the cell is a mammalian cell. In some embodiments, the cell is a human cell.

### Applications

In some aspects, the disclosure relates to a method of delivering WAEVs displaying an antigenic peptide, comprising: delivering at least one of any of the fusion proteins of the disclosure, at least one of any of the isolated nucleic acids of the disclosure, at least one of any of the WAEVs of the disclosure, and/or at least one of any of the WAEV-producing cells of the disclosure, wherein the extracellular protein of the fusion protein comprises an antigenic peptide.

Some aspects of this invention provide a method of delivering an extracellular domain (e.g., antigen), for example, by delivering a WAEV comprising a fusion protein comprising a WW-containing domain, transmembrane domain, and extracellular domain to a target cell. The target cell can be contacted with the WAEV in different ways. For example, a target cell may be contacted directly with a WAEV, including but not necessarily limited to, an isolated WAEV from a microvesicle-producing cell. The contacting can be done in vitro by administering the WAEV, fusion protein, and/or isolated nucleic acid, to the target cell in a culture dish, or in vivo by administering the WAEV, fusion protein, isolated nucleic acid, and/or a microvesicle-producing cell comprising a fusion protein and/or isolated nucleic acid to a subject. Alternatively, the target cell can be contacted with a microvesicle producing cell as described herein, either directly or indirectly, for example, in vitro by co-culturing the target cell and the microvesicle producing cell, or in vivo by administering a microvesicle producing cell to a subject harboring the target cell. Accordingly, the method may include contacting the target cell with a microvesicle, for example, a WAEV, as described herein. The target cell may be contacted with a microvesicle-producing cell, either directly or indirectly as described herein, or with an isolated microvesicle, wherein the produced or isolated microvesicle has a lipid bilayer, a SCAMP3 protein or variant thereof, and an extracellular domain.

It should be appreciated that the target cell may be of any origin. For example, the target cell may be a human cell. The target cell may be a mammalian cell. Some non-limiting examples of a mammalian cell include a mouse cell, a rat cell, hamster cell, a rodent cell, and a nonhuman primate cell. It should also be appreciated that the target cell may be of any cell type. For example the target cell may be a stem cell, which may include embryonic stem cells, induced pluripotent stem cells (iPS cells), fetal stem cells, cord blood stem cells, or adult stem cells (i.e., tissue specific stem cells). In other cases, the target cell may be any differentiated cell type found in a subject. In some embodiments, the target cell is a cell in vitro, and the method includes administering the microvesicle to the cell in vitro, or co-culturing the target cell with the microvesicle-producing cell in vitro. In some embodiments, the target cell is a cell in a subject, and the method comprises administering the microvesicle or the microvesicle-producing cell to the subject. In some embodiments, the subject is a mammalian subject, for example, a rodent, a mouse, a rat, a hamster, or a non-human primate. In some embodiments, the subject is a human subject.

In some embodiments, the target cell is a pathological cell. In some embodiments, the target cell is cell having, at risk of having, or suspected of having a disease or disorder. In some embodiments, the target cell is cell having, at risk of having, or suspected of having been exposed, or of being exposed to a pathogen (e.g., virus, bacteria). In some embodiments, the microvesicle is associated with presenting an antigen, or fragment thereof, to the cellular machinery of the target cell (e.g., immune cells).

In some embodiments, the microvesicles (e.g., WAEVs), fusion proteins, and/or isolated nucleic acids of the disclosure are used to provoke an immune response in a subject. Accordingly, in some embodiment, the microvesicles (e.g., WAEVs), fusion proteins, and/or isolated nucleic acids of the disclosure are administered to the subject. In some embodiment, the microvesicles (e.g., WAEVs), fusion proteins, and/or isolated nucleic acids of the disclosure comprise an extracellular domain comprising an antigen, or fragment thereof, which provokes, or is intended to provoke, an immune response to the antigen, or fragment thereof. In some embodiments, the antigen is a viral antigen or a bacterial antigen. In some embodiments, the administration disclosed as part of any of the methods disclosed herein, is in an effective amount.

For example, without limitation, the extracellular domain may contain an antigen, or fragment thereof, from a virus. In some embodiments, the virus may be a respiratory virus. In some embodiments, the respiratory virus is selected from the group consisting of: adenovirus (ADV); influenza virus, human bocavirus (HBoV); human coronavirus (HCoV); human metapneumovirus (HMPV); human parainfluenza virus (HPIV); human respiratory syncytial virus (HRSV); human rhinovirus (HRV); severe acute respiratory syndrome coronavirus (SARS-CoV); and Middle East Respiratory Syndrome coronavirus (MERS-CoV).

In some embodiments, a WAEV, fusion protein, and/or isolated nucleic acid having, or encoding, an extracellular domain as described herein may be administered to a subject.

In some embodiments, the subject is mammalian. In some embodiments, the subject is human.

### EXAMPLES

### Example 1

Described herein is a method of creating a novel type of extracellular vesicles (EVs) termed “WAEVs” (for WW-domain-Activated Extracellular Vesicles). WAEVs are produced via a fusion of WW-domains to a transmembrane segment that is linked to an extracellular domain, for example an extracellular peptide. Demonstrated herein is a method of making WAEVs in which the extracellular protein is a protein of the SARS-CoV-2 virus, including but not necessarily limited to the M protein, the E protein, or the S protein. WAEVs are distinct from classical exosomes as they are not enriched in one or more exosomal markers such as CD63, CD81, CD9, and/or PTGFRN. WAEVs are also distinct from ARRDC1-mediated microvesicles (ARMMs) despite the fact that ARMM budding is enhanced by WW-domain containing proteins. WAEV budding does not require ARRDC1 and is not enhanced by ARRDC1 overexpression. Proteomics analysis of WAEVs identified SCAMP3 (secretory carrier-associated membrane protein 3) as one potential mediator of WAEVs budding. SCAMP3 contains both PPXY (SEQ ID NO: 22) and PSAP (SEQ ID NO: 17) motifs. These motifs are elements in the ARRDC1 protein that are required for ARMMs budding. Without wishing to be bound by any theory, it is possible that the fusion protein comprising a WW-containing domain interacts with the PPXY (SEQ ID NO: 22) motif SCAMP3, which subsequently recruited TSG101 via PSAP (SEQ ID NO: 17) motif to the cell membrane to drive the budding of WAEVs. WAEVs are EV forms that is distinct from exosomes and ARMMs. WAEVs may be useful for, among other things: 1) displaying antigens to SARS-COV-2 virus for vaccine development

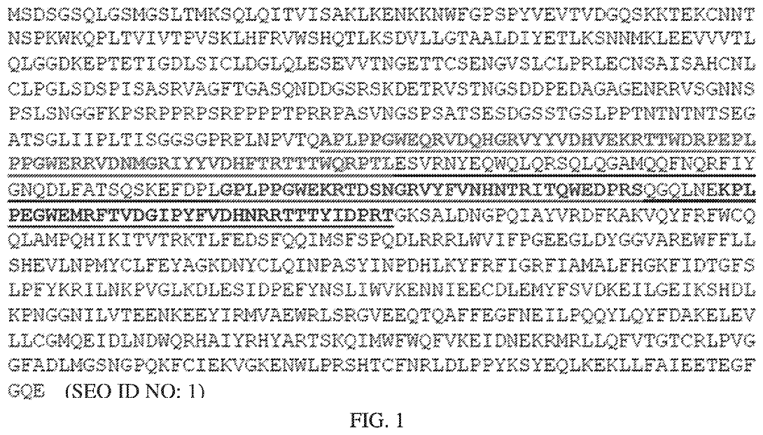

Direct fusion of a transmembrane-containing proteins to ARRDC1 seemingly abolished the budding activity of ARRDC1, thereby making it difficult to display peptides on ARMMSs. Since WW-containing domain proteins such as ITCH interact with ARRDC1 and can be recruited into ARMMs (Nabhan 2012, PNAS; Wang 2017 Nature Communications), it was determined whether the WW-containing domains (SEQ ID NO: 2-5) of the ITCH protein (SEQ ID NO: 1; see FIG. 1) could be used to display peptides onto the surface of extracellular vesicles.

It was determined that WAEVs could be used for presenting SARS-CoV-2 proteins, including but not necessarily limited to the M protein, the E protein, and the S protein.

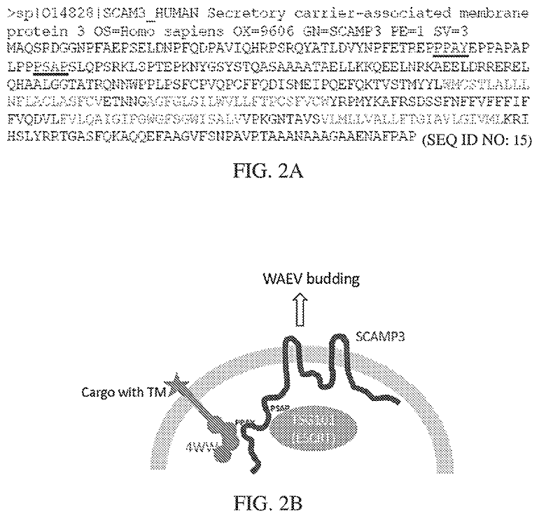

Without wanting to be bound by any theory, it is possible that one or more of the 20 enriched proteins may drive the biogenesis of WAEVs. Any potential candidate to carry out WAEV-budding function should 1) contain a PPXY (SEQ ID NO: 22) to interact with the WW-containing domain and 2) localize to the plasma membrane. Analysis of the common 20 enriched WAEV proteins identified SCAMP3 (secretory carrier-associated membrane protein 3) as a potential candidate. SCAMP3 is an integral membrane protein that has four transmembrane domains and contains a PPAY (SEQ ID NO: 16) motif at its N-terminal cytosolic segment (FIG. 2A). In addition, SCAMP3 has a PSAP (SEQ ID NO: 17) motif that is known to interact with TSG101 — the ESCRT I complex protein required for budding of ARMMs as well as other multivesicular bodies. Thus, SCAMP3 shares both PPXY (SEQ ID NO: 22) and PSAP (SEQ ID NO: 17) motif with ARRDC1 but differs from ARRDC1 in that SCAMP3 is integrated in the plasma membrane via its transmembrane domains whereas ARRDC1 transiently associates with plasma membrane via its arrestin domain. Based on these observations, it was proposed that a fusion protein with a WW-containing domain (such a fusion protein with transmembrane domain fused to a cargo domain) can interact with the PPXY (SEQ ID NO: 22) motif of SCAMP3, which can subsequently recruit TSG10S1 via the PSAP (SEQ ID NO: 17) motif to the cell membrane to drive the budding of WAEVs (see FIG. 2B).

### Example 2

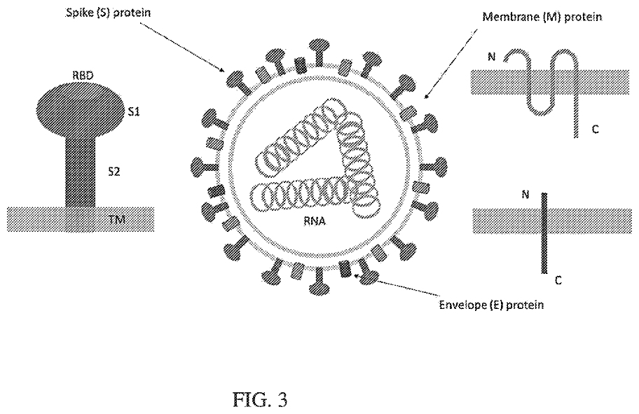

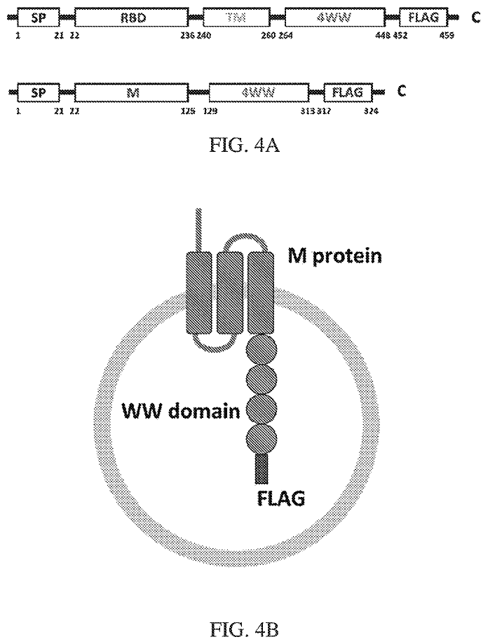

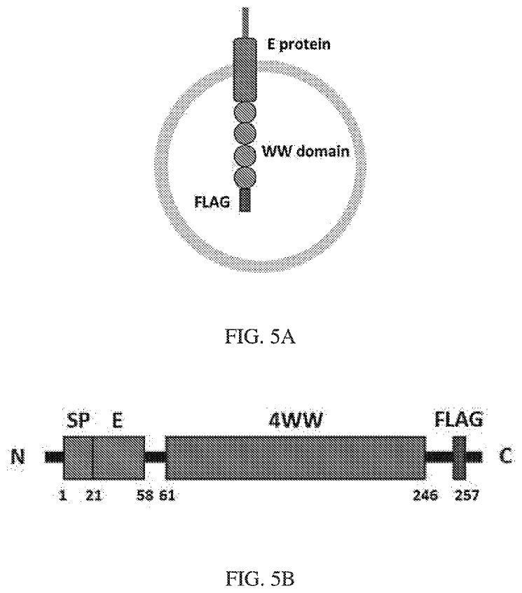

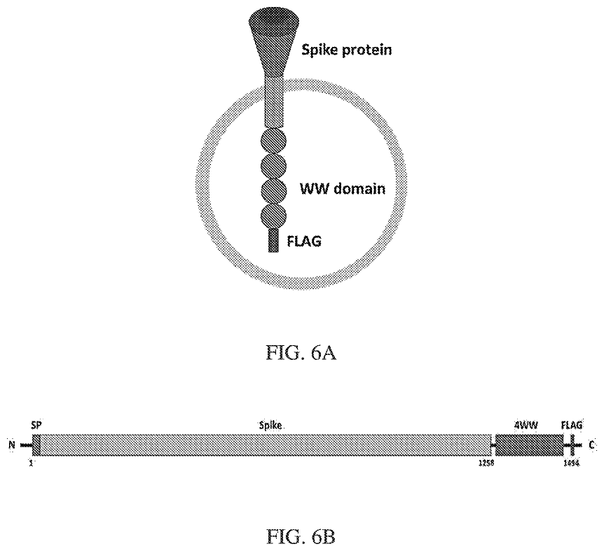

The WW domain constructs were tested to determine that viral proteins from SARS-CoV-2 could be presented on the surface of WAEVs. SARS-CoV-2 virus has several viral proteins on its surface. They include the spike (S) protein, the membrane (M) protein and the envelope (E) protein (see FIG. 3). Fusion constructs were made with the WW-containing domain fused to either RBD (with the transmembrane domain) or the M protein, the E protein, or the S protein (see FIGS. 4A, 5B, and 6B). The M-WW construct contains, at the C-terminus, 4WW domains from the ITCH protein, and at the C-terminus, the extracellular domain and transmembrane domain (TM) of the M protein. (see FIG. 4B). The E-WW construct contains, at the C-terminus, 4WW domains from the ITCH protein, and at the C-terminus, the extracellular domain and transmembrane domain (TM) of the E protein (see FIG. 5A). The S-WW construct contains, at the C-terminus, 4WW domains from the ITCH protein, and at the C-terminus, the full length of the S protein with its transmembrane domain (see FIG. 6B). All the constructs also contain a signal peptide (sp) to facilitate the processing and trafficking of the transmembrane fusion protein.

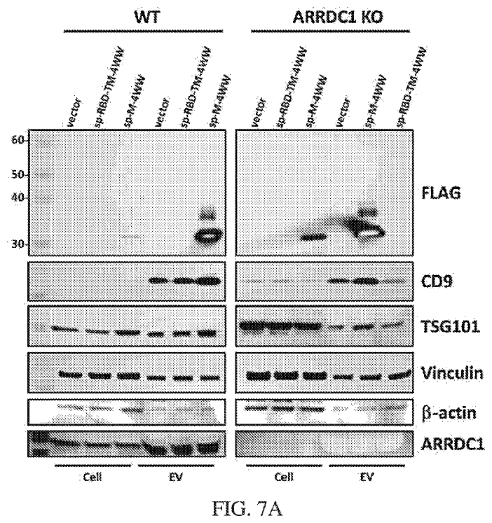

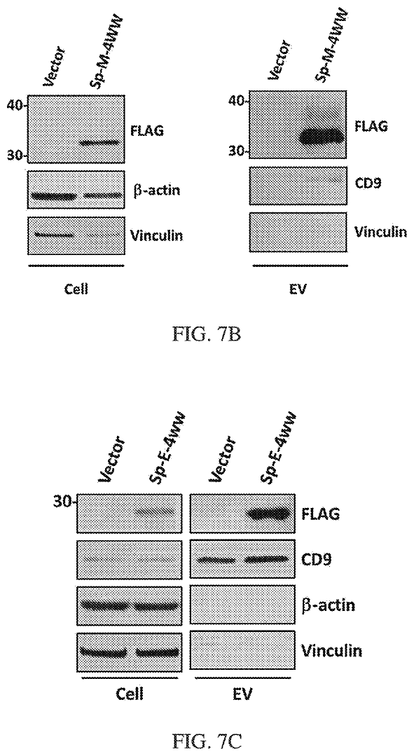

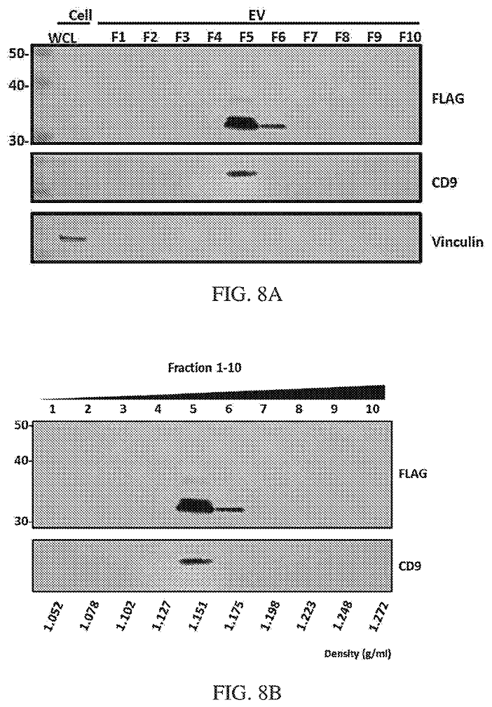

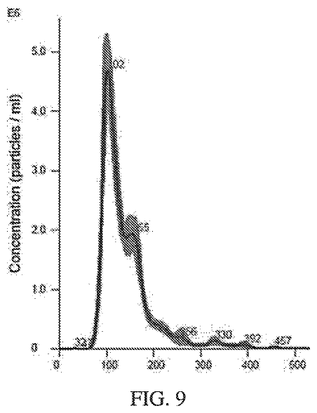

It was tested whether the fusion proteins are secreted into EVs, indicating that WAEVs could be used for budding of viral antigens of SARS-CoV-2. Both the receptor binding domain (RBD) of the spike protein and the membrane (M) protein of SARS-CoV-2 were tested. When transfected into HEK293T WT or ARRDC1 knockout cells, M-4WW fusion protein was expressed in cells and robustly budded out into EVs (see FIG. 7A). The particular RBD fusion protein failed to express in HEK293T cells. The M-4WW WAEVs were then purified using the density gradient ultracentrifugation. The peak fraction of M-4WW WAEVs seemed to overlap with that of exosomes (as indicated by the exosomal marker CD9) (see FIG. 8A-B).

To determine whether M-4WW WAEVs elicits anti-SARS-CoV2 antibody, mice are immunized with the WAEVs. Animal models are also used to determine the potential of M-4WW WAEVs in protecting against SARS-CoV-2 infection.

### Exemplary Sequences

The following Table exhibits some exemplary sequences as disclosed by the instant Specification, but is not limiting. This Specification includes a Sequence Listing submitted concurrently herewith as a text file in ASCII format. The Sequence Listing and all of the information contained therein are expressly incorporated herein and constitute part of the instant Specification as filed.

| Sequences* | Description** |
|---|---|
| <SEQ ID NO: 1> | |
| MSDSGSQLGSMGSLTMKSQLQITVISAKLKENKKNWFG | |
| PSPYVEVTVDGQSKKTEKCNNTNSPKWKQPLTVIVTPV | |
| SKLHFRVWSHQTLKSDVLLGTAALDIYETLKSNNMKLE | |
| EVVVTLQLGGDKEPTETIGDLSICLDGLQLESEVVTNG | |
| ETTCSENGVSLCLPRLECNSAISAHCNLCLPGLSDSPI | |
| SASRVAGFTGASQNDDGSRSKDETRVSTNGSDDPEDAG | |
| AGENRRVSGNNSPSLSNGGFKPSRPPRPSRPPPPTPRR | |
| PASVNGSPSATSESDGSSTGSLPPTNTNTNTSEGATSG | |
| LIIPLTISGGSGPRPLNPVTQAPLPPGWEQRVDQHGRV | |
| YYVDHVEKRTTWDRPEPLPPGWERRVDNMGRIYYVDHF | |
| TRTTTWQRPTLESVRNYEQWQLQRSQLQGAMQQFNQRF | |
| IYGNQDLFATSQSKEFDPLGPLPPGWEKRTDSNGRVYF | |
| VNHNTRITQWEDPRSQGQLNEKPLPEGWEMRFTVDGIP | |
| YFVDHNRRTTTYIDPRTGKSALDNGPQIAYVRDFKAKV | |
| QYFRFWCQQLAMPQHIKITVTRKTLFEDSFQQIMSFSP | |
| QDLRRRLWVIFPGEEGLDYGGVAREWFFLLSHEVLNPM | |
| YCLFEYAGKDNYCLQINPASYINPDHLKYFRFIGRFIA | |
| MALFHGKFIDTGFSLPFYKRILNKPVGLKDLESIDPEF | |
| YNSLIWVKENNIEECDLEMYFSVDKEILGEIKSHDLKP | |
| NGGNILVTEENKEEYIRMVAEWRLSRGVEEQTQAFFEG | |
| FNEILPQQYLQYFDAKELEVLLCGMQEIDLNDWQRHAI | |
| YRHYARTSKQIMWFWQFVKEIDNEKRMRLLQFVTGTCR | |
| LPVGGFADLMGSNGPQKFCIEKVGKENWLPRSHTCFNR | |
| LDLPPYKSYEQLKEKLLFAIEETEGFGQE | |
| <SEQ ID NO: 2> | |
| APLPPGWEQRVDQHGRVYYVDHVEKRTTWDRPEPLPPG | |
| WERRVDNMGRIYYVDHFTRTTTWQRPTL | |
| <SEQ ID NO: 3> | |
| ESVRNYEQWQLQRSQLQGAMQQFNQRFIYGNQDLFATS | |
| QSKEFDPL | |
| <SEQ ID NO: 4> | |
| GPLPPGWEKRTDSNGRVYFVNHNTRITQWEDPRS | |
| <SEQ ID NO: 5> | |
| QGQLNE | |
| <SEQ ID NO: 6> | |
| KPLPEGWEMRFTVDGIPYFVDHNRRTTTYIDPRT | |
| <SEQ ID NO: 7> | |
| MADSNGTITVEELKKLLEQWNLVIGFLFLTWICLLQFA | |
| YANRNRFLYIIKLIFLWLLWPVTLACFVLAAVYRINWI | |
| TGGIAIAMACLVGLMWLSYFIASFRLFARTRSMWSFNP | |
| ETNILLNVPLHGTILTRPLLESELVIGAVILRGHLRIA | |
| GHHLGRCDIKDLPKEITVATSRTLSYYKLGASQRVAGD | |
| SGFAAYSRYRIGNYKLNTDHSSSSDNIALLVQ | |
| <SEQ ID NO: 8> | |
| MADSNGTITVEELKKLLEQWNLVIGFLFLTWICLLQFA | |
| YANRNRFLYIIKLIFLWLLWPVTLACFVLAAVYRINWI | |
| TGGIAIAMACLVGLMWLSYFIASFRLFA | |
| <SEQ ID NO: 9> | |
| MYSFVSEETGTLIVNSVLLFLAFVVFLLVTLAILTALR | |
| LCAYCCNIVNVSLVKPSFYVYSRVKNLNSSRVPDLLV | |
| <SEQ ID NO: 10> | |
| MFVFLVLLPLVSSQCVNLTTRTQLPPAYTNSFTRGVYY | |
| PDKVFRSSVLHSTQDLFLPFFSNVTWFHAIHVSGTNGT | |
| KRFDNPVLPFNDGVYFASTEKSNIIRGWIFGTTLDSKT | |
| QSLLIVNNATNVVIKVCEFQFCNDPFLGVYYHKNNKSW | |
| MESEFRVYSSANNCTFEYVSQPFLMDLEGKQGNFKNLR | |
| EFVFKNIDGYFKIYSKHTPINLVRDLPQGFSALEPLVD | |
| LPIGINITRFQTLLALHRSYLTPGDSSSGWTAGAAAYY | |
| VGYLQPRTFLLKYNENGTITDAVDCALDPLSETKCTLK | |
| SFTVEKGIYQTSNFRVQPTESIVRFPNITNLCPFGEVF | |
| NATRFASVYAWNRKRISNCVADYSVLYNSASFSTFKCY | |
| GVSPTKLNDLCFTNVYADSFVIRGDEVRQIAPGQTGKI | |
| ADYNYKLPDDFTGCVIAWNSNNLDSKVGGNYNYLYRLF | |
| RKSNLKPFERDISTEIYQAGSTPCNGVEGFNCYFPLQS | |
| YGFQPTNGVGYQPYRVVVLSFELLHAPATVCGPKKSTN | |
| LVKNKCVNFNFNGLTGTGVLTESNKKFLPFQQFGRDIA | |
| DTTDAVRDPQTLEILDITPCSFGGVSVITPGTNTSNQV | |
| AVLYQDVNCTEVPVAIHADQLTPTWRVYSTGSNVFQTR | |
| AGCLIGAEHVNNSYECDIPIGAGICASYQTQTNSPRRA | |
| RSVASQSIIAYTMSLGAENSVAYSNNSIAIPTNFTISV | |
| TTEILPVSMTKTSVDCTMYICGDSTECSNLLLQYGSFC | |
| TQLNRALTGIAVEQDKNTQEVFAQVKQIYKTPPIKDFG | |
| GFNFSQILPDPSKPSKRSFIEDLLFNKVTLADAGFIKQ | |
| YGDCLGDIAARDLICAQKFNGLTVLPPLLTDEMIAQYT | |
| SALLAGTITSGWTFGAGAALQIPFAMQMAYRFNGIGVT | |
| QNVLYENQKLIANQFNSAIGKIQDSLSSTASALGKLQD | |
| VVNQNAQALNTLVKQLSSNFGAISSVLNDILSRLDKVE | |
| AEVQIDRLITGRLQSLQTYVTQQLIRAAEIRASANLAA | |
| TKMSECVLGQSKRVDFCGKGYHLMSFPQSAPHGVVFLH | |
| VTYVPAQEKNFTTAPAICHDGKAHFPREGVFVSNGTHW | |
| FVTQRNFYEPQIITTDNTFVSGNCDVVIGIVNNTVYDP | |
| LQPELDSFKEELDKYFKNHTSPDVDLGDISGINASVVN | |
| IQKEIDRLNEVAKNLNESLIDLQELGKYEQYIKWPWYI | |
| WLGFIAGLIAIVMVTIMLCCMTSCCSCLKGCCSCGSCC | |
| KFDEDDSEPVLKGVKLHYT | |
| <SEQ ID NO: 15> | |
| MAQSRDGGNPFAEPSELDNPFQDPAVIQHRPSRQYATLDVYNPFET | |
| REPPPAYEPPAPAPLPPPSAPSLQPSRKLSPTEPKNYGSYSTQASA | |
| AAATAELLKKQEELNRKAEELDRRERELQHAALGGTATRQNNWPPL | |
| PSFCPVQPCFFQDISMEIPQEFQKTVSTMYYLWMCSTLALLLNFLA | |
| CLASFCVETNNGAGFGLSILWVLLFTPCSFVCWYRPMYKAFRSDSS | |
| FNFFVFFFIFFVQDVLFVLQAIGIPGWGFSGWISALVVPKGNTAVS | |
| VLMLLVALLFTGIAVLGIVMLKRIHSLYRRTGASFQKAQQEFAAGV | |
| FSNPAVRTAAANAAAGAAENAFRAP | |
| <SEQ ID NO: 16> | |
| PPAY | |
| <SEQ ID NO: 17> | |
| PSAP | |
| <SEQ ID NO: 18> | |
| WMCSTLALLLNFLACLASFCV | |
| <SEQ ID NO: 19> | |
| AGFGLSILWVLLFTPCSFVCW | |
| <SEQ ID NO: 20> | |
| FVLQAIGIPGWGFSGWISALV | |
| <SEQ ID NO: 21> | |
| VLMLLVALLFTGIAVLGIVML | |
| <SEQ ID NO: 22> | |
| PPXY | |
| <SEQ ID NO: 61> | Fusion 1 |
| METDTLLLWVLLLWVPGSTGDMFVFLVLLPLVSSQCVNLTTRTQLP | |
| PAYTNSFTRGVYYPDKVFRSSVLHSTQDLFLPFFSNVTWFHAIHVS | |
| GTNGTKRFDNPVLPFNDGVYFASTEKSNIIRGWIFGTTLDSKTQSL | |
| LIVNNATNVVIKVCEFQFCNDPFLGVYYHKNNKSWMESEFRVYSSA | |
| NNCTFEYVSQPFLMDLEGKQGNFKNLREFVFKNIDGYFKIYSKHTP | |
| INLVRDLPQGFSALEPLVDLPIGINITRFQTLLALHRSYLTPGDSS | |
| SGWTAGAAAYYVGYLQPRTFLLKYNENGTITDAVDCALDPLSETKC | |
| TLKSFTVEKGIYQTSNFRVQPTESIVRFPNITNLCPFGEVFNATRF | |
| ASVYAWNRKRISNCVADYSVLYNSASFSTFKCYGVSPTKLNDLCFT | |
| NVYADSFVIRGDEVRQIAPGQTGKIADYNYKLPDDFTGCVIAWNSN | |
| NLDSKVGGNYNYLYRLFRKSNLKPFERDISTEIYQAGSTPCNGVEG | |
| FNCYFPLQSYGFQPTNGVGYQPYRVVVLSFELLHAPATVCGPKKST | |
| NLVKNKCVNFNFNGLTGTGVLTESNKKFLPFQQFGRDIADTTDAVR | |
| DPQTLEILDITPCSFGGVSVITPGTNTSNQVAVLYQDVNCTEVPVA | |
| IHADQLTPTWRVYSTGSNVFQTRAGCLIGAEHVNNSYECDIPIGAG | |
| ICASYQTQTNSPRRARSVASQSIIAYTMSLGAENSVAYSNNSIAIP | |
| TNFTISVTTEILPVSMTKTSVDCTMYICGDSTECSNLLLQYGSFCT | |
| QLNRALTGIAVEQDKNTQEVFAQVKQIYKTPPIKDFGGFNFSQILP | |
| DPSKPSKRSFIEDLLFNKVTLADAGFIKQYGDCLGDIAARDLICAQ | |
| KFNGLTVLPPLLTDEMIAQYTSALLAGTITSGWTFGAGAALQIPFA | |
| MQMAYRFNGIGVTQNVLYENQKLIANQFNSAIGKIQDSLSSTASAL | |
| GKLQDVVNQNAQALNTLVKQLSSNFGAISSVLNDILSRLDKVEAEV | |
| QIDRLITGRLQSLQTYVTQQLIRAAEIRASANLAATKMSECVLGQS | |
| KRVDFCGKGYHLMSFPQSAPHGVVFLHVTYVPAQEKNFTTAPAICH | |
| DGKAHFPREGVFVSNGTHWFVTQRNFYEPQIITTDNTFVSGNCDVV | |
| IGIVNNTVYDPLQPELDSFKEELDKYFKNHTSPDVDLGDISGINAS | |
| VVNIQKEIDRLNEVAKNLNESLIDLQELGKYEQYIKWPWYIWLGFI | |
| AGLIAIVMVTIMLCCMTSCCSCLKGCCSCGSCCKFDEDDSEPVLKG | |
| VKLHYTGGGPLPPGWEQRVDQHGRVYYVDHVEKRTTWDRPEPLPPG | |
| WERRVDNMGRIYYVDHFTRTTTWQRPTLESVRNYEQWQLQRSQLQG | |
| AMQQFNQRFIYGNQDLFATSQSKEFDPLGPLPPGWEKRTDSNGRVY | |
| FVNHNTRITQWEDPRSQGQLNEKPLPEGWEMRFTVDGIPYFVDHNR | |
| RTTTYIDPRTGGGDYKDDDDK | |
| <SEQ ID NO: 62> | Fusion 2 |
| METDTLLLWVLLLWVPGSTGDIVRFPNITNLCPFGEVFNATRFASV | |
| YAWNRKRISNCVADYSVLYNSASFSTFKCYGVSPTKLNDLCFTNVY | |
| ADSFVIRGDEVRQIAPGQTGKIADYNYKLPDDFTGCVIAWNSNNLD | |
| SKVGGNYNYLYRLFRKSNLKPFERDISTEIYQAGSTPCNGVEGFNC | |
| YFPLQSYGFQPTNGVGYQPYRVVVLSFELLHAPATVCGPKKSTNLG | |
| GGWYIWLGFIAGLIAIVMVTIMLGGGPLPPGWEQRVDQHGRVYYVD | |
| HVEKRTTWDRPEPLPPGWERRVDNMGRIYYVDHFTRTTTWORPTLE | |
| SVRNYEQWQLQRSQLQGAMQQFNQRFIYGNQDLFATSQSKEFDPLG | |
| PLPPGWEKRTDSNGRVYFVNHNTRITQWEDPRSQGQLNEKPLPEGW | |
| EMRFTVDGIPYFVDHNRRTTTYIDPRTGGGDYKDDDDK | |
| <SEQ ID NO: 63> | Fusion 3 |
| METDTLLLWVLLLWVPGSTGDMADSNGTITVEELKKLLEQWNLVIG | |
| FLFLTWICLLQFAYANRNRFLYIIKLIFLWLLWPVTLACFVLAAVY | |
| RINWITGGIAIAMACLVGLMWLSYFIASFRLFAGGGPLPPGWEQRV | |
| DQHGRVYYVDHVEKRTTWDRPEPLPPGWERRVDNMGRIYYVDHFTR | |
| TTTWQRPTLESVRNYEQWQLQRSQLQGAMQQFNQRFTYGNQDLFAT | |
| SQSKEFDPLGPLPPGWEKRTDSNGRVYFVNHNTRITQWEDPRSQGQ | |
| LNEKPLPEGWEMRFTVDGIPYFVDHNRRTTTYIDPRTGGGDYKDDD | |
| DK | |
| <SEQ ID NO: 64> | Fusion 4 |
| METDTLLLWVLLLWVPGSTGDMYSFVSEETGTLIVNSVLLFLAFVV | |
| FLLVTLAILTALGGGPLPPGWEQRVDQHGRVYYVDHVEKRTTWDRP | |
| EPLPPGWERRVDNMGRIYYVDHFTRTTTWQRPTLESVRNYEQWQLQ | |
| RSQLQGAMQQFNQRFIYGNQDLFATSQSKEFDPLGPLPPGWEKRTD | |
| SNGRVYFVNHNTRITQWEDPRSQGQLNEKPLPEGWEMRFTVDGIPY | |
| FVDHNRRTTTYIDPRTGGGDYKDDDDK | |
| <SEQ ID NO: 65> | FLAG Domain |
| DYKDDDDK | |
| <SEQ ID NO: 66> | Signal Peptide (SP) |
| METDTLLLWVLLLWVPGSTGD | Domain |
| <SEQ ID NO: 67> | Spike Domain |
| MFVFLVLLPLVSSQCVNLTTRTQLPPAYTNSFTRGVYYPDKVFRSS | |
| VLHSTQDLFLPFFSNVTWFHAIHVSGTNGTKRFDNPVLPFNDGVYF | |
| ASTEKSNIIRGWIFGTTLDSKTQSLLIVNNATNVVIKVCEFQFCND | |
| PFLGVYYHKNNKSWMESEFRVYSSANNCTFEYVSQPFLMDLEGKQG | |
| NFKNLREFVFKNIDGYFKIYSKHTPINLVRDLPQGFSALEPLVDLP | |
| IGINITRFQTLLALHRSYLTPGDSSSGWTAGAAAYYVGYLQPRTFL | |
| LKYNENGTITDAVDCALDPLSETKCTLKSFTVEKGIYQTSNFRVQP | |
| TESIVRFPNITNLCPFGEVFNATRFASVYAWNRKRISNCVADYSVL | |
| YNSASFSTFKCYGVSPTKLNDLCFTNVYADSFVIRGDEVRQIAPGQ | |
| TGKIADYNYKLPDDFTGCVIAWNSNNLDSKVGGNYNYLYRLFRKSN | |
| LKPFERDISTEIYQAGSTPCNGVEGFNCYFPLQSYGFQPTNGVGYQ | |
| PYRVVVLSFELLHAPATVCGPKKSTNLVKNKCVNFNFNGLTGTGVL | |
| TESNKKFLPFQQFGRDIADTTDAVRDPQTLEILDITPCSFGGVSVI | |
| TPGTNTSNQVAVLYQDVNCTEVPVAIHADQLTPTWRVYSTGSNVFQ | |
| TRAGCLIGAEHVNNSYECDIPIGAGICASYQTQTNSPRRARSVASQ | |
| SIIAYTMSLGAENSVAYSNNSIAIPTNFTISVTTEILPVSMTKTSV | |
| DCTMYICGDSTECSNLLLQYGSFCTQLNRALTGIAVEQDKNTQEVF | |
| AQVKQIYKTPPIKDFGGFNFSQILPDPSKPSKRSFIEDLLFNKVTL | |
| ADAGFIKQYGDCLGDIAARDLICAQKFNGLTVLPPLLTDEMIAQYT | |
| SALLAGTITSGWTFGAGAALQIPFAMQMAYRFNGIGVTQNVLYENQ | |
| KLIANQFNSAIGKIQDSLSSTASALGKLQDVVNQNAQALNTLVKQL | |
| SSNFGAISSVLNDILSRLDKVEAEVQIDRLITGRLQSLQTYVTQQL | |
| IRAAEIRASANLAATKMSECVLGQSKRVDFCGKGYHLMSFPQSAPH | |
| GVVFLHVTYVPAQEKNFTTAPAICHDGKAHFPREGVFVSNGTHWFV | |
| TQRNFYEPQIITTDNTFVSGNCDVVIGIVNNTVYDPLQPELDSFKE | |
| ELDKYFKNHTSPDVDLGDISGINASVVNIQKEIDRLNEVAKNLNES | |
| LIDLQELGKYEQYIKWPWYIWLGFIAGLIAIVMVTIMLCCMTSCCS | |
| CLKGCCSCGSCCKFDEDDSEPVLKGVKLHYT | |
| <SEQ ID NO: 68> | RBD Domain |
| IVRFPNITNLCPFGEVFNATRFASVYAWNRKRISNCVADYSVLYNS | |
| ASFSTFKCYGVSPTKLNDLCFTNVYADSFVIRGDEVRQIAPGQTGK | |
| IADYNYKLPDDFTGCVIAWNSNNLDSKVGGNYNYLYRLFRKSNLKP | |
| FERDISTEIYQAGSTPCNGVEGFNCYFPLQSYGFQPTNGVGYQPYR | |
| VVVLSFELLHAPATVCGPKKSTNL | |
| <SEQ ID NO: 69> | TM Domain Domain |
| WYIWLGFIAGLIAIVMVTIML | |
| <SEQ ID NO: 70> | M protein domain |
| MADSNGTITVEELKKLLEQWNLVIGFLFLTWICLLQFAYANRNRFL | |
| YIIKLIFLWLLWPVTLACFVLAAVYRINWITGGIAIAMACLVGLMW | |
| LSYFIASFRLFA | |
| <SEQ ID NO: 71> | E protein Domain |
| MYSFVSEETGTLIVNSVLLFLAFVVFLLVTLAILTAL | |
| <SEQ ID NO: 72> | 4WW Domain |
| PLPPGWEQRVDQHGRVYYVDHVEKRTTWDRPEPLPPGWERRVDNMG | |
| RIYYVDHFTRTTTWQRPTLESVRNYEQWQLQRSQLQGAMQQFNQRF | |
| IYGNQDLFATSQSKEFDPLGPLPPGWEKRTDSNGRVYFVNHNTRIT | |
| QWEDPRSQGQLNEKPLPEGWEMRFTVDGIPYFVDHNRRTTTYIDPR | |
| T | |
| <SEQ ID NO: 73> | Gly-Linker Domain |
| GGG | |

*Unless otherwise specified, nucleic acid sequences are described 5′ to 3′ and amino acid sequences are described N-terminus to C-terminus
**‘NT’ denotes a nucleic acid sequence; ‘AA’ denotes an amino acid sequence. In the absence of an identifier (e.g., NT, AA), the skilled artisan will readily be able to discern nucleic acid sequences from amino acid sequences by their constituent components. For example, a nucleic acid will only contain those identifiers associated in the art with ribonucleic acid or deoxyribonucleic acid components (e.g., A, C, G, T, U, or other modified base (i.e., nucleotide)) whereas amino acid sequences will contain those identifiers associated in the art with amino acid components (e.g., A, C, D, E, F, G, H, I, K, L, M, N, P, Q, R, S, T, V, W, Y, or other modified amino acid).

### General Techniques

The practice of the subject matter of the disclosure will employ, unless otherwise indicated, conventional techniques of molecular biology (including recombinant techniques), microbiology, cell biology, biochemistry and immunology, which are within the skill of the art. Such techniques are explained fully in the literature, such as, but without limiting, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### EQUIVALENTS AND SCOPE

It is to be understood that this disclosure is not limited to any or all of the particular embodiments described expressly herein, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents (i.e., any lexicographical definition in the publications and patents cited that is not also expressly repeated in the disclosure should not be treated as such and should not be read as defining any terms appearing in the accompanying claims). If there is a conflict between any of the incorporated references and this disclosure, this disclosure shall control. In addition, any particular embodiment of this disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

In the claims articles such as “a,” “an,” and “the” may mean one or more than one unless indicated to the contrary or otherwise evident from the context. Wherever used herein, a pronoun in a gender (e.g., masculine, feminine, neuter, other, etc . . . ) the pronoun shall be construed as gender neutral (i.e., construed to refer to all genders equally) regardless of the implied gender unless the context clearly indicates or requires otherwise. Wherever used herein, words used in the singular include the plural, and words used in the plural includes the singular, unless the context clearly indicates or requires otherwise. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

What is claimed is:

1. A fusion protein comprising:
(a) a WW-containing domain;
(b) a transmembrane domain; and
(c) an extracellular domain, wherein the extracellular domain is a coronavirus antigen domain.

2. The fusion protein of claim 1, wherein the coronavirus antigen domain is a SARS-COV-2 antigen domain.

3. The fusion protein of any of claims 1-2, wherein the fusion protein does not comprise an arrestin domain containing protein 1 (ARRDC1).

4. The fusion protein of any one of claims 1-3, wherein the WW-containing domain comprises at least one WW domain. The fusion protein of any one of claims 1-4, wherein the WW-containing domain comprises at least two WW domain.

6. The fusion protein of any one of claims 1-5, wherein the WW-containing domain comprises at least three WW domain.

7. The fusion protein of any one of claims 1-6, wherein the WW-containing domain comprises at least four WW domain.

8. The fusion protein of any of claims 4-7, wherein the WW-containing domain is a NEDD4 E3 ligase domain.

9. The fusion protein of claim 8, wherein the NEDD4 E3 ligase is selected from the group consisting of ITCH, NEDD4, NEDD4 L, WWP1, WWP2, Smurf1, Smurf2, BUL1, and NEDL2.

10. The fusion protein of claim 8, wherein the NEDD4 E3 ligase is the ITCH protein.

11. The fusion protein of any one of claims 1-10, wherein the WW-containing domain comprises a sequence having at least 95% identity to the sequence of SEQ ID NO: 1.

12. The fusion protein of any one of claims 1-11, wherein the WW-containing domain comprises the sequence of SEQ ID NO: 1.

13. The fusion protein of any one of claim 1-12, wherein the transmembrane domain comprises a coronavirus transmembrane domain.

14. The fusion protein of any of claims 1-13, wherein the coronavirus transmembrane domain comprises a SARS-COV-2 transmembrane domain.

15. The fusion protein of claim 14, wherein the coronavirus transmembrane domain comprises a SARS-COV-2 transmembrane domain.

16. The fusion protein of claim 15, wherein the coronavirus transmembrane domain comprises a SARS-COV-2 M protein transmembrane domain.

17. The fusion protein of claim 15, wherein the coronavirus transmembrane domain comprises a SARS-COV-2 E protein transmembrane domain.

18. The fusion protein of claim 15, wherein the coronavirus transmembrane domain comprises a SARS-COV-2 S protein transmembrane domain.

19. The fusion protein of any one of claims 1-13, wherein the transmembrane domain comprises a sequence having at least 95% identity to the sequence of SEQ ID NO:7.

20. The fusion protein of any one of claims 1-13, wherein the transmembrane domain comprises a sequence having at least 95% identity to the sequence of SEQ ID NO: 9.

21. The fusion protein of any one of claims 1-13, wherein the transmembrane domain comprises a sequence having at least 95% identity to the sequence of SEQ ID NO:10.

22. The fusion protein of any one of claims 1-13, wherein the transmembrane domain comprises the sequence of SEQ ID NO: 7.

23. The fusion protein of any one of claims 1-13, wherein the transmembrane domain comprises the sequence of SEQ ID NO: 9.

24. The fusion protein of any one of claims 1-13, wherein the transmembrane domain comprises the sequence of SEQ ID NO: 10.

25. The fusion protein of any one of claims 1-24, wherein the extracellular domain is a coronavirus antigen domain.

26. The fusion protein of claim 25, wherein the coronavirus antigen domain is a SARS-COV-2 antigen domain.

27. The fusion protein of claim 26, wherein the SARS-CoV-2 antigen domain is an M protein extracellular domain.

28. The fusion protein of claim 26, wherein the SARS-CoV-2 antigen domain is an E protein extracellular domain.

29. The fusion protein of claim 26, wherein the SARS-CoV-2 antigen domain is an S protein extracellular domain.

30. The fusion protein of any one of claims 1-24, wherein the extracellular domain comprises a sequence having at least 95% identity to the sequence of SEQ ID NO: 7.

31. The fusion protein of any one of claims 1-24, wherein the extracellular domain comprises a sequence having at least 95% identity to the sequence of SEQ ID NO: 9.

32. The fusion protein of any one of claims 1-24, wherein the extracellular domain comprises a sequence having at least 95% identity to the sequence of SEQ ID NO: 10.

33. The fusion protein of any one of claims 1-24, wherein the extracellular domain comprises the sequence of SEQ ID NO: 7.

34. The fusion protein of any one of claims 1-24, wherein the extracellular domain comprises the sequence of SEQ ID NO: 9.

35. The fusion protein of any one of claims 1-24, wherein the extracellular domain comprises the sequence of SEQ ID NO: 10.

36. The fusion protein of any one of claims 1-35, further comprising a signal peptide.

37. The fusion protein of claim 1, wherein the fusion protein comprises a sequence having at least 95% identity to the sequence of SEQ ID NO: 61.

38. The fusion protein of claim 1, wherein the fusion protein consists of a sequence having at least 95% identity to the sequence of SEQ ID NO: 61.

39. The fusion protein of claim 1, wherein the fusion protein comprises the sequence of SEQ ID NO: 61.

40. The fusion protein of claim 1, wherein the fusion protein consists of the sequence of SEQ ID NO: 61.

41. The fusion protein of claim 1, wherein the fusion protein comprises a sequence having at least 95% identity to the sequence of SEQ ID NO: 62.

42. The fusion protein of claim 1, wherein the fusion protein consists of a sequence having at least 95% identity to the sequence of SEQ ID NO: 62.

43. The fusion protein of claim 1, wherein the fusion protein comprises the sequence of SEQ ID NO: 62.

44. The fusion protein of claim 1, wherein the fusion protein consists of the sequence of SEQ ID NO: 62.

45. The fusion protein of claim 1, wherein the fusion protein comprises a sequence having at least 95% identity to the sequence of SEQ ID NO: 63.

46. The fusion protein of claim 1, wherein the fusion protein consists of a sequence having at least 95% identity to the sequence of SEQ ID NO: 63.

47. The fusion protein of claim 1, wherein the fusion protein comprises the sequence of SEQ ID NO: 63.

48. The fusion protein of claim 1, wherein the fusion protein consists of the sequence of SEQ ID NO: 63.

49. The fusion protein of claim 1, wherein the fusion protein comprises a sequence having at least 95% identity to the sequence of SEQ ID NO: 64.

50. The fusion protein of claim 1, wherein the fusion protein consists of a sequence having at least 95% identity to the sequence of SEQ ID NO: 64.

51. The fusion protein of claim 1, wherein the fusion protein comprises the sequence of SEQ ID NO: 64.

52. The fusion protein of claim 1, wherein the fusion protein consists of the sequence of SEQ ID NO: 64.

53. An isolated nucleic acid encoding a fusion protein of any one of claims 1-52.

54. The isolated nucleic acid of claim 53, operably linked to a promoter.

55. The isolated nucleic acid of claim 54, wherein the promoter is a constitutive promoter, an inducible promoter, or a tissue specific promoter.

56. The isolated nucleic acid of any of claims 53-55, comprising at least one additional regulatory sequence.

57. A WW protein domain activated extracellular vesicle (WAEV), comprising:
(a) a lipid bilayer; and
(b) the fusion protein of any one of claims 1-52.

58. The WAEV of claim 57, further comprising SCAMPS.

59. The WAEV of claim 57 or 58, wherein the fusion protein does not comprise at least one of the following exosomal markers: CD63; CD81, CD9, and PTGFRN.

60. The WAEV of any one of claims 57-59, wherein the fusion protein does not comprise any of the following exosomal markers: CD63; CD81, CD9, and PTGFRN.

61. A WAEV-producing cell, comprising:
(a) a recombinant expression construct encoding the fusion protein of any one of claims 1-52 under the control of a heterologous promoter.

62. A WAEV-producing cell, comprising:
(a) the isolated nucleic acid of any one of claims 53-56.

63. A method of delivering WAEVs displaying a coronavirus antigenic peptide, comprising: delivering the fusion protein of any one of claims 1-52, the isolated nucleic acid of any one of claims 53-56, the WAEV of any one of claims 57-60, or the WAEV-producing cell of any one of claims 61-62 to a subject, wherein the extracellular protein of the fusion protein comprises an HIV antigenic peptide.

64. The method of claim 63, wherein the subject is mammalian.

65. The method of claim 64 or 65, wherein the subject is human.

66. A kit comprising one or more of the fusion protein of any one of claims 1-52, the isolated nucleic acid of any one of claims 53-56, the WAEV of any one of claims 57-60, or the WAEV-producing cell of any one of claims 61-62.
